(19) United States
(12) Patent Application Publication
(10) Pub. No.: US 2011/0264136 A1
(43) Pub. Date: Oct. 27, 2011

(54) SURGICAL INSTRUMENT

(76) Inventors: Seung Wook Choi, Gyeonggi-do (KR); Jong Seok Won, Gyeonggi-do (KR); Jae Sun Lee, Gyeonggi-do (KR)

(21) Appl. No.: 13/129,334

(22) PCT Filed: Dec. 8, 2009
(86) PCT No.: PCT/KR2009/007290

§ 371 Date: May 13, 2011

(30) Foreign Application Priority Data
Dec. 12, 2008 (KR) 10-2008-0126415
Dec. 30, 2008 (KR) 10-2008-0136840
Dec. 30, 2008 (KR) 10-2008-0136859
Jan. 21, 2009 (KR) 10-2009-0004872

(51) Int. Cl. A61B 17/28

(52) U.S. Cl. 606/205

(57) ABSTRACT

A surgical instrument is disclosed. The surgical instrument, which has an effector for engaging the surgical site joined to one end and a driving part for operating the effector joined to the other end, includes: a first shaft, which has one end joined with the driving part, and which extends along a first lengthwise direction; and a second shaft, which extends along a second lengthwise direction that forms a particular angle with the first shaft, and which has one end joined with the other end of the first shaft such that the second shaft is rotatable about an axis following the second lengthwise direction. Thus, it is possible to conduct surgery using several of such surgical instruments without having the instruments obstruct one another, and the surgical instrument can be made to have different usage modes according to what length it is set to.

### TECHNICAL FIELD

The present invention relates to a medical apparatus, more particularly to a surgical instrument.

### BACKGROUND ART

In the field of medicine, surgery refers to a procedure in which a medical apparatus is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars. Thus, current methods of surgery that involve making an incision in the skin and inserting only a medical apparatus, such as a laparoscope, a surgical instrument, and a microscope, for example, or those that involve the use of surgical robots are currently regarded as popular alternatives.

A set of surgical robots may include a master robot, which is manipulated by the doctor to generate and transmit the necessary signals, and a slave robot, which receives the signals from the master robot to actually apply the manipulation to the patient. The master robot and the slave robot can be arranged in the operating room as an integrated unit or as separate devices.

A slave robot may be equipped with a robot arm to make manipulations for surgery, while an instrument may be mounted on the front end of the robot arm. As illustrated in FIG. 1, a conventional instrument 54 for mounting on a robot arm may include a driving part 108, a shaft 102 extending from the driving part 108, and a forceps-like effector 112 mounted on the far end 106 of the shaft 102 that is to be inserted into the surgical site.

On a bottom surface of this type of conventional instrument 54, a multiple number of drive wheels (not shown) may be joined. Wires connected to different portions of the effector 112 may be respectively pulley-joined with the drive wheels, so that the rotations of the drive wheels may apply tension to the wires, causing the portions of the effector 112 to move and thus grab or cut the surgical site.

Although this surgical instrument may be suitable for procedures such as laparoscopic surgery that involve making multiple incisions, it may not be so suitable for surgical procedures in which only one incision is made. Procedures for single port access (SPA) surgery or microsurgery, for example, include inserting a vision system (a laparoscope, microscope, etc.) and a surgical instrument all through one hole. In current microsurgery procedures, such as for replantation surgery, spine surgery, brain surgery, etc., a microscope and a surgical instrument may be inserted after making just one hole or a slit having a length of 1 to 2 cm, instead of making multiple incisions. A conventional surgical instrument (including those for robot surgery) may not provide a desired level of freedom in such microsurgery or SPA surgery procedures. That is, if several of these surgical instruments are placed through one hole or a small slit, the instruments' housings 108, i.e. the couplers, may obstruct one another, making it very inconvenient to use conventional surgical instruments in these types of surgeries.

Also, according to the related art, it can be difficult for a user to utilize a surgical instrument in a convenient and efficient manner, as the user is unable to bend or unbend the shaft 102 at will.

A conventional instrument 54 may have an adapter part that joins to the robot arm. The driving forces may be transferred from the robot arm to rotate the driving wheels (not shown) that are pulley-joined by wires to the respective parts of the effector 112, and as a result, the parts of the effector 112 may be moved. In the case of a manually operated instrument, the surgeon may manipulate the driving part to move the effector, when holding or cutting the surgical site.

However, for a type of surgery that is performed with just one incision made in the surgical site, such as single port access (SPA) surgery and microsurgery, etc., a conventional surgical instrument may not be used with a high level of freedom, since a laparoscope and the instrument may all be inserted through the one incision for surgery.

Among existing types of surgery, a laparotomy is a surgical procedure in which the skin of the face or the abdomen is cut open and an internal organ, etc., is treated, reconstructed, or excised. When conducting a laparotomy, an incision is made in the skin and a particular amount of space is formed between the skin and the tissue, with the surgical operation performed within this space. As this may increase scars and prolong the healing period, laparoscopic surgery is recently gaining attention as an alternative.

In laparoscopic surgery, a small incision is made in the surgical site of the patient, through which a laparoscope is inserted, so that the surgery may be conducted while observing the surgical site within the abdominal cavity. Laparoscopic surgery is widely used in various fields of medicine, including internal medicine, surgery, urology, gynecology, and obstetrics. When conducting laparoscopic surgery, a surgical instrument may be used in which an effector is joined to the far end of a shaft that extends from the driving part. The effector portion of the surgical instrument may be inserted into the surgical site, and as the surgeon manipulates the driving part, the effector may function as a surgical tool, such as a set of scissors or forceps, etc., to perform various actions required for surgery.

The surgical instrument can be manipulated manually by the surgeon, or in the case of robot surgery, can be mounted onto the end portion of a robot arm, to be manipulated according to driving forces transferred from the robot arm.

However, in the case of a conventional instrument used for laparoscopic surgery, there is a limit to how small the diameter of the shaft can be made, and a trocar may have to be inserted beforehand into the surgical site through which to insert the instrument. In order to insert the trocar, the skin of the patient may have to be cut by a certain amount.

The information in the background art described above was obtained by the inventors for the purpose of developing the present invention or was obtained during the process of developing the present invention. As such, it is to be appreciated that this information did not necessarily belong to the public domain before the patent filing date of the present invention.

### DISCLOSURE OF INVENTION

### Technical Problem

An aspect of the present invention is to provide a surgical instrument that can be used for surgery in multiple numbers without obstructing one another.

Also, an aspect of the present invention is to provide a surgical instrument that provides various usage modes according to what length it is set to.

An aspect of the present invention is to provide a flexible surgical instrument that can be bent or unbent arbitrarily by the user and can be used in multiple numbers without obstructing one another.

An aspect of the present invention is to provide a surgical instrument that can be used in multiple numbers simultaneously without interfering with or obstructing one another and can be manipulated intuitively as if the surgeon were using one's own hands.

Also, an aspect of the present invention is to provide a surgical instrument, and a method of setting the instrument, with which the diameter of the surgical instrument can be minimized, so that the incision made in the skin is of a size that does not require suturing.

Other technical problems addressed by the present invention will be readily understood from the descriptions that follow.

### Solution to Problem

One aspect of the present invention provides a surgical instrument that has an effector for engaging the surgical site joined to one end and a driving part for operating the effector joined to the other end. This surgical instrument includes: a first shaft, which has one end joined with the driving part, and which extends along a first lengthwise direction; and a second shaft, which extends along a second lengthwise direction that forms a particular angle with the first shaft, and which has one end joined with the other end of the first shaft such that the second shaft is rotatable about an axis following the second lengthwise direction.

The second shaft can be bearing-joined with the first shaft such that the second shaft is able to rotate, and the first shaft and second shaft can have different lengths.

Also, the driving part can be a coupler, which may include a driving wheel that is operated by a driving force transferred from a surgical robot arm.

Another aspect of the present invention provides a robotic surgical instrument for mounting on the front end of a surgical robot arm that includes an actuator. This robotic surgical instrument includes: a coupler, which includes a driving wheel that is operated by a driving force transferred from the actuator; a first shaft, which has one end joined with the coupler, and which extends along a first lengthwise direction; a second shaft, which has one end joined with the other end of the first shaft, and which extends along a second lengthwise direction that forms a particular angle with the first shaft, where the second shaft is rotatable about an axis following the second lengthwise direction; and an effector joined to the other end of the second shaft for inserting into the body of a surgery patient.

The second shaft can be orthogonal to the first shaft, and the second shaft can be configured to rotate in correspondence with an operation of the driving wheel.

Also, the second shaft can be rotated by a wire that is joined with the driving wheel and the second shaft, while the effector can be manipulated in correspondence with an operation of the driving wheel.

The first shaft and the second shaft can have different lengths. For example, the length of the second shaft can be shorter than that of the first shaft, and the second shaft may be inserted into the body of a surgery patient, or alternatively, the length of the second shaft can be longer than that of the first shaft.

The second shaft can be bearing-joined with the first shaft such that the second shaft is able to rotate. The robotic surgical instrument can further include a rotatable roller part, which supports a wire that connects the driving wheel and the effector, at a joint part where the first shaft and the second shaft are joined.

The driving wheel can be shaped as a circular disk and may clutch onto the actuator to receive a driving force transferred from the actuator.

Also, the robotic surgical instrument can further include a bending part, which is capable of bending, positioned between the second shaft and the effector. A wire can be joined to the driving wheel that applies a tensional force to bend the bending part in a particular direction.

Still another aspect of the present invention provides a flexible surgical instrument that has an effector for engaging the surgical site joined to one end and a driving part for operating the effector joined to the other end. This flexible surgical instrument includes: a shaft, which has one end joined with the driving part, extends along a particular lengthwise direction, and includes a bending part that is capable of bending; and a cover part, which holds the bending part of the shaft.

The distance between one end of the shaft and the bending part can be different from the distance between the other end of the shaft and the bending part, and the bending part can be bent by a force applied by a user.

Another aspect of the present invention provides a flexible surgical instrument that has an effector for engaging the surgical site joined to one end and a driving part for operating the effector joined to the other end. This flexible surgical instrument includes: a shaft, which has one end joined with the driving part, extends along a particular lengthwise direction, and has a flexible form; and a cover part, which holds a bent portion of the shaft to maintain the bent angle of the shaft.

The shaft can be made from a flexible material or can be made as a flexible structure. In such cases, the shaft may be a corrugated tube made of metal or synthetic resin.

The cover part can be flexible or rigid, and can be detachable and attachable in relation to the shaft.

The driving part can be a coupler that includes a driving wheel operated by a driving force transferred from a surgical robot arm, to which the flexible surgical instrument is joined.

Still another aspect of the present invention provides a flexible robotic surgical instrument for mounting on the front end of a surgical robot arm that includes an actuator. This flexible robotic surgical instrument includes: a coupler, which includes a driving wheel operated by a driving force transferred from the actuator; a shaft, which has one end joined with the coupler, extends along a particular lengthwise direction, and is capable of bending; a cover part, which holds a bent portion of the shaft to maintain the bent angle of the shaft; and an effector joined to the other end of the shaft for inserting into the body of a surgery patient.

Here, the shaft can include a first bending part having a bendable form, and the cover part can hold the first bending part. The shaft can be made from a flexible material or can be made as a flexible structure.

Here, the shaft can be a corrugated tube made of metal or synthetic resin, and the cover part can include an angle adjusting part for adjusting the bending angle in correspondence with the bent portion of the shaft.

Furthermore, the angle adjusting part can be a stopper or a screw, and the shaft can be rotatable about an axis following the lengthwise direction.

The distance between one end of the shaft and the bent portion can be different from the distance between the other end of the shaft and the bent portion.

The driving wheel can be shaped as a circular disk and can be configured to clutch onto the actuator to receive a driving force transferred from the actuator.

The flexible robotic surgical instrument can also further include a second bending part, which has a bendable form, positioned between the shaft and the effector. A wire can be joined to the driving wheel to apply a tensional force that bends the second bending part in a particular direction.

Yet another aspect of the present invention provides a medical trocar that includes: a tube-shaped cannula; and a trocar housing, which includes a housing hole connected to an opening of the cannula, joined to one end of the cannula. The cannula is flexible, so that a surgical instrument having a bendable shaft may be inserted through the cannula.

The trocar housing can include a drive valve, to which a wire may be joined that applies a tensional force to bend the cannula in a particular direction. The drive valve can be moved by a driving wheel, which in turn may be operated by a driving force transferred from a surgical robot arm to which the surgical instrument is joined. A multiple number of holes can be perforated in the trocar through which to insert a multiple number of instruments.

Another aspect of the present invention provides a surgical instrument that includes:

a driving part; a shaft joined to the driving part that extends along one direction and has an elbow formed in the middle; and an effector joined to the far end of the shaft that operates in correspondence with a user manipulation on the driving part, where the shaft can be configured to curve at the elbow.

The elbow can include a hinge axis, formed on one side as seen from a cross section of the shaft, and an expandable part, formed on the other side of the cross section of the shaft, where the shaft can be configured to curve at the hinge axis in a direction that compresses the expandable part. The expandable part can include an elastic body that applies an elastic force in a direction that expands the expandable to straighten the shaft or compresses the expandable part to curve the shaft. In this case, the driving part can include a driver, with a wire connecting the driver with a particular point in a vicinity of the elbow, and the shaft can be curved at the elbow by manipulating the driver to apply a tensional force on the wire.

There can be a multiple number of elbows formed in the shaft, and the elbows can be formed to curve the shaft in opposite directions, so that the effector may move closer to the driving part as the shaft is curved.

The shaft can include a core and a guide member, where the core may be made from a flexible material, and the guide member may surround the core, with the elbow formed in a portion of the guide member. Thus, the core can be curved as the guide member is curved. In this case, the guide member can be used as a surgical trocar.

A wire can be connected to a point near the guide member, and by applying a tensional force on the wire, the guide member may be curved at the elbow. A driving wheel may be joined to the guide member, and the wire may be connected to the driving wheel, where a tensional force can be applied on the wire by manipulating the driving wheel. In this case, the driving part can include a driver, with the driving wheel connected to the driver, to be manipulated in linkage with a manipulation of the driver.

The wire can be installed exposed at a surface of the shaft, and the wire may be pulled out of the shaft as a tensional force is applied on the wire to curve the shaft. In this case, the shaft can have a cylindrical shape, and the wire can form a portion of the perimeter of the shaft. Also, the shaft can be formed with a channel processed in its cross section to hold the wire.

The driving part can be coupled to a surgical robot arm to be manipulated by a driving force transferred from the robot, or alternatively, can be formed as a handle to be manually manipulated by a user.

Still another aspect of the present invention provides a master interface for a surgical robot. The master interface is mounted on a master robot and enables a user to conduct robotic surgery by manipulating a surgical instrument mounted on a slave robot connected to the master robot. This master interface includes an elbow handle that generates a particular manipulation signal for operating the instrument, where an elbow is formed in a shaft of the instrument, the shaft is configured to curve at the elbow, and the elbow handle is configured to generate the manipulation signal for curving the shaft. In this case, the elbow handle can be worn on an elbow of a user, to be operated in accordance with the movement of the user's elbow.

Yet another aspect of the present invention provides a method of driving a surgical instrument mounted on a slave robot by connecting the slave robot to a master robot and manipulating the master robot. This method includes: generating a particular manipulation signal in correspondence with the movement of an elbow handle, which is included on the master robot, and which is worn on an elbow of a user; converting the manipulation signal into a driving signal that corresponds to a curving operation of a shaft of the instrument; and transmitting the driving signal to the slave robot. After the transmitting, the method can further include: curving the shaft to correspond with a movement of the elbow of the user, using the driving signal.

The general and specific aspects above can be implemented as a system, method, or a computer program, or as any combination of systems, methods, and computer programs.

Another aspect of the present invention provides a surgical instrument that includes:

a driving part; a multiple number of rods joined to the driving part that extend along a lengthwise direction; and an effector detachably joined to a far end of the rods that operates in correspondence with a manipulation on the driving part. The multiple number of rods can form a set, which may be used as a shaft.

Still another aspect of the present invention provides a surgical instrument that includes: a driving part; a multiple number of rods joined to the driving part that extend along a lengthwise direction and form a set serving as a shaft; and an effector that is joined to a far end of the rods and configured to operate in correspondence with a manipulation on the driving part. The effector can be detachably joined to the far end of the set of rods. In forming a set, the multiple number of rods can be fastened together at one or more points along the middle portion, or be twisted around one another. One or more of the rods can connect the driving part with the effector, serving to support and secure the effector in a particular position.

A multiple number of drivers, which may be joined respectively to one end of the rods, can be installed in the driving part, and the rods can be operated in correspondence with a manipulation on the respective drivers. The effector can include a multiple number of interlocking parts that are detachably joined with the other end of the rods, respectively, and the effector can be operated according to an operation of the rods while the rods are joined to the interlocking parts.

The interlocking parts are included in numbers that enable the effector to be operated with n (n is a natural number) degrees of freedom, the drivers are included in numbers that enable the driving part to be manipulated with n degrees of freedom, and the plurality of interlocking parts are joined by the plurality of rods with the plurality of drivers respectively in a corresponding manner.

In this case, an interlocking part and the other end of a rod can be shaped as a pair of linking devices that mate with each other. The multiple linking devices formed on the multiple interlocking parts and the other ends of the multiple rods can be formed with different shapes, so that each of the interlocking parts may be joined only with the other end of the rod which mates with the interlocking part.

A needle for invading can be mounted on the other end of the rod, while the rod can include a conductive element and an insulative element that coats and surrounds the conductive element, and the needle can be electrically connected with the conductive element. A cable can be used to supply electrical power to a tip part of the effector, which may then be used as an electrosurgical device.

The effector can be formed in a size capable of passing through a trocar inserted in the surgical site.

Another aspect of the present invention provides a method of setting a surgical instrument that includes: providing an effector, which includes a multiple number of interlocking parts, and which is configured to operate in accordance with a manipulation on the interlocking parts; providing a multiple number of rods, where each of the rods has one end joined to the driving part, the rods are configured to operate in correspondence with a manipulation on the driving part, and the rods each have a linking device formed on the other end in a shape that mates with a respective interlocking part; joining the other ends of the rods with the mating interlocking parts, respectively; and operating the effector by manipulating the driving part.

Additional aspects, features, and advantages, other than those described above, will be obvious from the claims and written description below.

### Advantageous Effects of Invention

Certain embodiments of the present invention make it possible to conduct surgery using several surgical instruments without having the instruments obstruct one another, and a surgical instrument can be made to have different usage modes according to what length it is set to.

Also, by forming an elbow in the shaft of a surgical instrument and enabling the shaft to bend according to a manipulation on the driving part, the shaft of the instrument can be made to perform articular movements similar to those of a wrist or an elbow. Thus, a surgeon may manipulate the instrument intuitively, just as if the surgeon were using his or her own hands.

Since the shaft of the instrument can be bent as necessary, several instruments can be inserted from different directions through a single insertion hole, and for each instrument, the shaft can be bent such that the effector faces a particular surgical site. Thus, even when using more than one instruments at once, the instruments may not interfere with or obstruct one another, and an effective mode of “minimally invasive surgery” can be implemented.

Also, by forming the effector and a rod to be attachable and detachable in relation to each other, and by forming a multiple number of rods as one set that can be used as a substitute for the shaft, it is possible to minimize the diameter of the instrument. Furthermore, by first inserting the effector into the surgical site, then invading the rod and joining the effector and the rod within the surgical site, it is possible to utilize the surgical instrument after making an incision in the surgical site of a size that does not require suturing.

### BRIEF DESCRIPTION OF DRAWINGS

FIG. 1 is a perspective view of a surgical instrument according to the related art.

FIG. 2 is a perspective view of a surgical instrument according to an embodiment of the present invention.

FIG. 3 is a perspective view illustrating the joint part of a surgical instrument according to an embodiment of the present invention.

FIG. 4 is a diagram illustrating the use of surgical instruments according to an embodiment of the present invention.

FIG. 5 is a diagram illustrating the use of surgical instruments according to another embodiment of the present invention.

FIG. 6 is a perspective view of a surgical instrument according to another embodiment of the present invention.

FIG. 7 is a perspective view illustrating the joint part and bending part of a surgical instrument according to another embodiment of the present invention.

FIG. 8 is a perspective view of a flexible surgical instrument according to an embodiment of the present invention.

FIG. 9 is a perspective view illustrating the bending part of a flexible surgical instrument according to an embodiment of the present invention.

FIG. 10 and FIG. 11 are diagrams illustrating the uses of flexible surgical instruments according to embodiments of the present invention.

FIG. 12 is a perspective view of a flexible surgical instrument according to another embodiment of the present invention.

FIG. 13 is a perspective view illustrating the bending part of a flexible surgical instrument according to another embodiment of the present invention.

FIG. 14 is a perspective view of a flexible surgical instrument according to another embodiment of the present invention.

FIG. 15 is a drawing illustrating a cover part of a flexible surgical instrument according to an embodiment of the present invention.

FIG. 16 is a drawing illustrating a linking structure between a flexible surgical instrument and a medical trocar according to an embodiment of the present invention.

FIG. 17 is a diagram schematically illustrating a surgical instrument according to an embodiment of the present invention.

FIG. 18 is a magnified view of the elbow portion of a surgical instrument according to an embodiment of the present invention.

FIG. 19 is a diagram illustrating the operation of a surgical instrument according to an embodiment of the present invention.

FIG. 20 is a diagram illustrating the operation of a surgical instrument according to another embodiment of the present invention.

FIG. 21 is a diagram illustrating the operation of a surgical instrument according to another embodiment of the present invention.

FIG. 22 is a diagram illustrating possible cross sections for the shaft of a surgical instrument according to an embodiment of the present invention.

FIG. 23 is a diagram illustrating the composition of a surgical robot according to an embodiment of the present invention.

FIG. 24 is a perspective view of a master interface for a surgical robot according to an embodiment of the present invention.

FIG. 25 is a flowchart illustrating a method of driving a surgical robot according to an embodiment of the present invention.

FIG. 26 is a diagram schematically illustrating a surgical instrument according to an embodiment of the present invention.

FIG. 27 is a lateral cross-sectional view of a set of rods according to an embodiment of the present invention.

FIG. 28 is a diagram schematically illustrating a surgical instrument according to another embodiment of the present invention.

FIG. 29 is a diagram schematically illustrating the driving part of a surgical instrument according to an embodiment of the present invention.

FIG. 30 is a diagram schematically illustrating the effector of a surgical instrument according to an embodiment of the present invention.

FIG. 31 is a diagram illustrating the operation of a surgical instrument according to an embodiment of the present invention.

FIG. 32 is a flowchart illustrating a method of setting a surgical instrument according to an embodiment of the present invention.

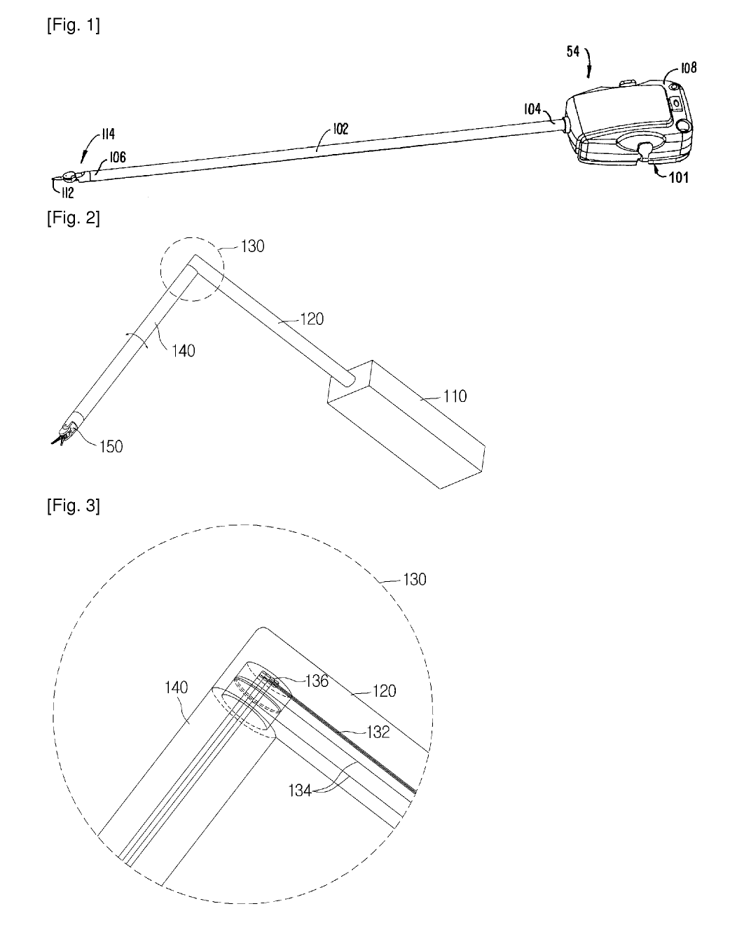

### MODE FOR THE INVENTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another. For example, a first component can be referred to as a second component without departing from the scope of claims of the present invention, and likewise, a second component can be referred to as a first component. If a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

FIG. 2 is a perspective view of a surgical instrument according to an embodiment of the present invention. Illustrated in FIG. 2 are a coupler 110, a first shaft 120, a joint part 130, a second shaft 140, and an effector 150.

A feature of this embodiment is to divide the shaft according to usage and function and extend each portion of the shaft in different directions, so that several surgical instruments may be used during actual surgery without obstructing one another, and the surgical procedures may be facilitated. That is, the shaft may be divided into a first shaft 120 and a second shaft 140, with the second shaft 140 extending in a direction different from that of the first shaft 120, so that the couplers 110 may not obstruct one another.

A surgical instrument according to this embodiment can be used in robotic surgery or in manual surgery. For the former case, the surgical instrument may be mounted on the front end of a surgical robot arm that is equipped with an actuator. Surgery may be conducted as a driving force transferred from the actuator operates a driving wheel (not shown) equipped in the coupler 110, causing the effector 150, which is connected with the driving wheel and is inserted into the body of the surgery patient, to perform a particular maneuver. The driving wheel can be shaped as a circular disk and may clutch onto the actuator to receive the driving force. The number of driving wheels can be determined in correspondence with the number of objects that require control. Details related to the driving wheel are obvious to the person skilled in the field of surgical instruments and thus will be omitted here.

For the latter case, the coupler 110 may be replaced by a particular driving part (not shown), for example an interface (shaped as sticks, buttons, forceps, levers, etc.) that can be directly manipulated by a doctor. Surgery may be performed when the doctor controls the interface, causing the effector 150, which is connected to the interface and inserted into the body of the surgery patient, to perform a particular maneuver. The following descriptions will be provided referring mainly to the former case.

The first shaft 120 may have one end joined with the coupler 110 and may extend along a first lengthwise direction to join with the second shaft 140. The second shaft 140 may have one end joined to the other end of the first shaft 120, may extend along a second lengthwise direction that forms a certain angle with the first shaft 120, and may be structured to be rotatable about an axis following the second lengthwise direction.

Here, the first lengthwise direction and the second lengthwise direction are different directions, and the angle formed by the two directions can be adjusted within a range that enables greater utility during actual surgery, for example, to 90 degrees. Since the first shaft 120 may extend along the first lengthwise direction and the second shaft 140 may extend along the second lengthwise direction, there is a lower risk that the couplers 110 will obstruct one another, when more than one of such surgical instruments are used in surgery. Therefore, the surgical instruments and robotic surgery can be applied even to microsurgery and SPA surgery procedures. In particular, if a surgical instrument structured as above, i.e. folded and extending along a first direction and a second direction, is joined with a robot arm, a greater level of freedom can be provided in terms of the direction in which the robot arm is installed and the direction in which the surgical instrument is extended, etc., making it possible to conceive new surgical techniques.

The first shaft 120 and the second shaft 140 may be joined together in such a way that the second shaft 140 is rotatable about the second lengthwise direction. For example, the first shaft 120 and the second shaft 140 can be bearing-joined with each other. Here, a bearing-joint refers to a joint that enables smooth rotational motion by reducing friction between the first shaft 120 and the second shaft 140.

Also, at the joint part, where the first shaft 120 joins the second shaft 140, a rotatable roller part can be included that supports a wire, which connects the driving wheel with the effector 150. That is, the wire connecting the driving wheel and the effector 150 can be bent at the joint part by the angle formed by the first lengthwise direction and the second lengthwise direction, and in this embodiment, a roller part can be included that facilitates the contracting and relaxing of the wire.

The wire can be divided mainly into two types: a wire for connecting the driving wheel and the effector 150 and a wire for connecting the driving wheel and the second shaft 140. The driving wheel may be divided into parts for controlling the movement of the effector 150 and parts for controlling the movement of the second shaft 140. Thus, the number of driving wheels can be determined in correspondence to the number of wires.

The effector 150 may be joined to the other end of the second shaft 140 and may be inserted into the body of the surgery patient. The effector 150 is the member that engages the surgical site during actual surgery. The effector 150 of the surgical instrument may include a pair of jaws, which may be joined to the far end of the second shaft 140 to perform a gripping or cutting movement. Also, the effector 150 can be formed such that the whole of the effector 150 is able to rotate in linkage with the rotation of the second shaft 140.

In this case, the driving wheels of the driving part can be pulley-joined with the pair of jaws. Various methods can be used for joining the driving wheels with the pair of jaws, such as joining a set of wires to each of the jaws or joining a set of wires to the pair of jaws, for example. Referring to the latter case, as the driving wheels are rotated, the driving forces may be transferred by way of the wires, so that the pair of jaws may perform a gripping or cutting movement. In moving the pair of jaws using a set of pulley-wires, the pair of jaws may be connected by gears, etc., and the pulley-wires can be joined to one of the pair of jaws or to a portion where the pair of jaws are joined, to transfer the driving forces. Of course, various other mechanisms can be applied in which a set of pulleys are used that enable the pair of jaws to perform a gripping movement.

FIG. 3 is a perspective view illustrating the joint part of a surgical instrument according to an embodiment of the present invention. Illustrated in FIG. 3 are a first shaft 120, a joint part 130, a first wire 132, a second wire 134, a roller part 136, and a second shaft 140.

The following descriptions will be provided with reference to a linking structure for wires that perform different functions and the joint part 130. As already described above, the wires can be divided into a first wire 132, which connects the driving wheel and the effector 150, and a second wire 134, which connects the driving wheel and the second shaft 140.

The first wire 132 may be joined at one end with the driving wheel and joined at the other end with the effector 150. The rotational movement of the driving wheel may cause the first wire 132 to undergo a contracting or relaxing motion, and in correspondence to this motion, the effector 150 may perform a particular operation, such as a gripping operation or a cutting operation.

The second wire 134 may be joined at one end with the driving wheel and joined at the other end with the second shaft 140. Various methods can be used by which the second wire 134 joins the second shaft 140, such as winding the second wire 134 around the second shaft 140, or affixing the second wire 134 to a certain point on the second shaft 140, for example. Of course, various other mechanisms for rotating the second shaft 140 using the second wire 134 can be applied to this embodiment.

As described above, the first shaft 120 and the second shaft 140 may be joined in such a way that the second shaft 140 is able to rotate about an axis following the second lengthwise direction. In this specification, this joining method will be referred to as a “bearing-joint.” Here, a bearing-joint not only includes linking structures such as a ball bearing, roller bearing, and plate bearing, but also encompasses various other linking structures, such as a screw-joint along an axis following the second lengthwise direction, and a linking structure that surrounds the perimeter of the second shaft 140 and uses a linking member that is held in an indentation formed in the perimeter. Of course, various other bearing-joints can be applied to this embodiment.

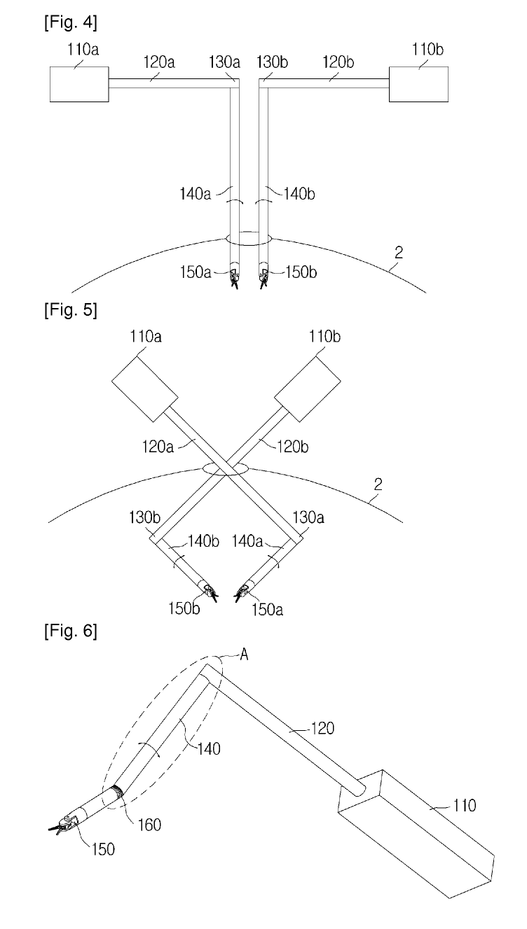

FIG. 4 and FIG. 5 are diagrams illustrating the uses of surgical instruments according to embodiments of the present invention. Illustrated in FIG. 4 and FIG. 5 are couplers 110a, 110b, first shafts 120a, 120b, joint parts 130a, 130b, second shafts 140a, 140b, effectors 150a, 150b, and a surgery patient 2.

In the example shown in FIG. 4, two surgical instruments according to this embodiment are inserted through one hole formed in the skin of the surgery patient 2, and the joint parts 130a, 130b are not inserted through the hole but are positioned outside the skin of the surgery patient 2. As the first shafts 120a, 120b, which join with the second shafts 140a, 140b, may extend in different directions, the couplers 110a, 110b may not obstruct each other. Here, the lengths of the second shafts 140a, 140b can be greater than the lengths of the first shafts 120a, 120b.

A laparoscope can additionally be inserted when conducting laparoscopic surgery, and a microscope can additionally be inserted when conducting microsurgery, but vision systems such as the laparoscope or microscope have been omitted from the drawings for more convenience. Also, a surgical operation may involve using a “flexible type” medical trocar, through which a bent surgical instrument may pass according to this embodiment. That is, when the surgical instrument is inserted into an abdominal cavity, a flexible type medical trocar can be used as necessary, as well as the “rigid type” medical trocar used in the related art.

In the example shown in FIG. 5, two surgical instruments according to this embodiment are inserted through one hole formed in the skin of the surgery patient 2, and the joint parts 130a, 130b are inserted through the hole, to be positioned inside the skin of the surgery patient 2. To conduct surgery more smoothly for this situation, the lengths of the second shafts 140a, 140b can be shorter than the lengths of the first shafts 120a, 120b. For example, when conducting SPA surgery, the effectors 150a, 150b can be moved towards the surgical site more easily and more efficiently, if the second shafts 140a, 140b are shorter than the first shafts 120a, 120b as in FIG. 5.

FIG. 6 is a perspective view of a surgical instrument according to another embodiment of the present invention. Illustrated in FIG. 6 are a coupler 110, a first shaft 120, a second shaft 140, an effector 150, and a bending part 160. The following descriptions will focus mainly on the differences from the previously described embodiment.

The bending part 160 may be positioned between the second shaft 140 and the effector 150 and may have a bendable structure. Here, to state that the bending part 160 may be positioned between the second shaft 140 and the effector 150 is intended to encompass not only those cases where the bending part 160, i.e. a bendable member, is formed over all of the length between the second shaft 140 and the effector 150, but also those cases where the bending part 160 is included at one end of the second shaft 140 and the effector 150 is joined to the far end after a particular length extending from the bending part 160, as illustrated in the drawing.

The bending part 160 may form a particular angle with the second lengthwise direction in which the second shaft 140 is extended, and may be formed as a bendable structure or from a bendable material. For example, the bending part 160 can be a structure that includes a multiple number of separate articulated parts and is bent when a certain amount of force is applied in a particular direction. Also, the bending part 160 can be made from a material high in plasticity, such as a synthetic resin tube.

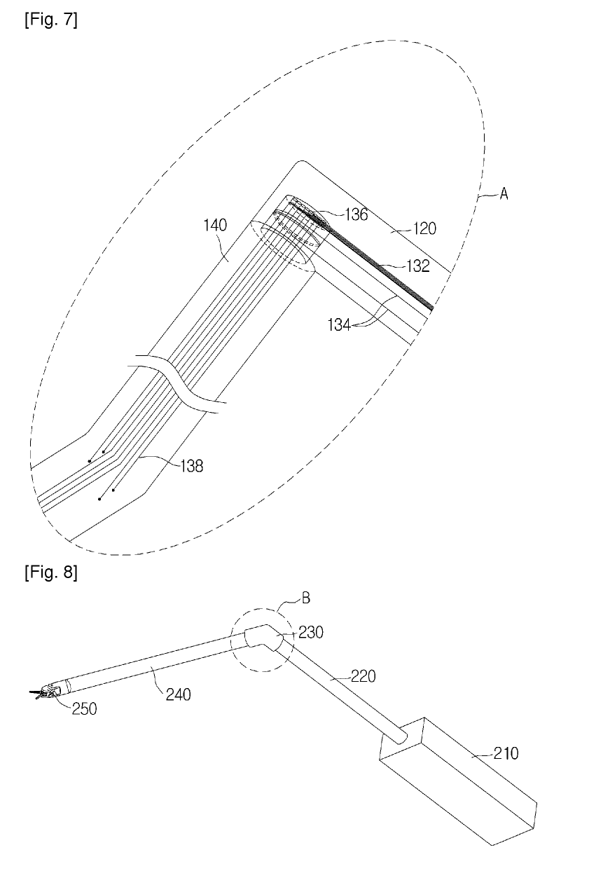

The bending part 160 may be controlled by the operation of driving wheels, and for this purpose, the bending part 160 and the driving wheels can be connected by wires. Referring to FIG. 7, which is a magnified view of area A, third wires 138 may connect the driving wheels with the bending part 160, whereby the movement of the bending part 160 can be controlled by the manipulation of the driving wheels. The third wires 138 may each have one end attached to one of four portions, respectively, within the bending part 160, for example in intervals of 90 degrees. The other ends of the third wires 138 may be joined to the driving wheels, and the rotational movements of the driving wheels may contract or relax the third wires 138 and thus adjust the tensional forces applied, so that the angle and direction in which the bending part 160 is bent may be determined accordingly. To implement such movements, additional driving wheels can be provided for manipulating the bending part 160. Of course, various other mechanisms for bending the bending part 160 using the third wires 138 can be applied to this embodiment.

Providing the surgical instrument with such a bending part 160 can increase the degree of freedom in controlling movements, so that the surgery may be conducted with greater convenience. That is, if bending parts 160 are included in the examples of FIG. 4 and FIG. 5, the effectors 150a, 150b may be positioned in the surgical site more conveniently and more efficiently.

Other details related to the surgical instrument according to an embodiment of the present invention or related to the surgical robot which the instrument may operate in linkage with, including, for example, detailed mechanical designs, common platform technology, such as the embedded system, O/S, etc., interface standardization technology, such as the communication protocol, I/O interface, etc., and component standardization technology, such as for actuators, batteries, cameras, sensors, etc., are obvious to those of ordinary skill in the field of art to which the present invention belongs and thus will be omitted here.

While the surgical instrument according to an embodiment of the present invention has been described above with reference to certain examples regarding the number and functions of the shafts, the present invention is not thus limited. Other compositions, in which the shaft is divided into smaller segments, or in which the operation method does not utilize wires, for example, can be encompassed by the scope of claims of the present invention if the overall actions and effects are substantially the same.

FIG. 8 is a perspective view of a flexible surgical instrument according to an embodiment of the present invention. Illustrated in FIG. 8 are a coupler 210, a shaft front part 220, a cover part 230, a shaft rear part 240, and an effector 250.

A feature of this embodiment is that the shaft can be bent by a force applied by the user, so that several surgical instruments may be used during actual surgery without obstructing one another, and the surgical procedures may be facilitated. That is, a first bending part may be provided in a certain position of the shaft, such as the middle position, for example. Then, during surgery, the user may bend this first bending part to a certain angle, and afterwards cover the first bending part with the cover part 230, so that the couplers 210 may not obstruct one another. The first bending part can be held in the cover part 230 to be used during surgery in a bent state.

The cover part 230 can be formed as a detachably attachable structure. For example, the cover part 230 can be shaped as a tube that is bent by a preset angle and can be made of two members that bisect the cross section of the tube. In this case, the user may bend the first bending part to a desired angle, select a cover part 230 that corresponds to the bent angle, and position the cover part 230 to cover the first bending part.

The cover part 230 can be of a flexible or a rigid form. In cases where the cover part 230 is flexible, the user may apply force on the first bending part and bend the shaft to a particular angle while the first bending part is held in the cover part 230. For this purpose, the cover part 230 can be made from a material that is bendable when an amount of force greater than a particular value is applied. In cases where the cover part 230 is rigid, the user may bend the first bending part to a particular angle, and then encase the first bending part with the unbendable cover part 230, so that this shape may be preserved.

According to another embodiment, the whole shaft can be made from a bendable material or made as a bendable structure, instead of having the first bending part in only a particular point of the shaft. In this case, a bent position can be referred to as the first bending part, and in order to maintain this bent shape, the first bending part can be held in the cover part 230.

A flexible surgical instrument according to this embodiment can be used in robotic surgery or in manual surgery. For the former case, the surgical instrument may be mounted on the front end of a surgical robot arm that is equipped with an actuator. Surgery may be conducted as a driving force transferred from the actuator operates a driving wheel (not shown) equipped in the coupler 210, causing the effector 250, which is connected with the driving wheel and is inserted into the body of the surgery patient, to perform a particular maneuver. The driving wheel can be shaped as a circular disk and may clutch onto the actuator to receive the driving force. The number of driving wheels can be determined in correspondence with the number of objects that require control. Details related to the driving wheel are obvious to the person skilled in the field of surgical instruments and thus will be omitted here.

For the latter case, the coupler 210 may be replaced by a particular driving part (not shown), for example an interface (shaped as sticks, buttons, forceps, levers, etc.) that can be directly manipulated by a doctor. Surgery may be performed when the doctor controls the interface, causing the effector 250, which is connected to the interface and inserted into the body of the surgery patient, to perform a particular maneuver. The following descriptions will be provided referring mainly to the former case.

The shaft front part 220 may have one end joined with the coupler 210 and may extend along a first lengthwise direction to join with the first bending part held in the cover part 230. The shaft rear part 240 may have one end joined with the first bending part and may extend along a second lengthwise direction that forms a certain angle with the shaft front part 220.

Here, the first lengthwise direction and the second lengthwise direction are different directions, and the angle formed by the two directions can be adjusted within a range that enables greater utility during actual surgery. Since the shaft front part 220 may extend along the first lengthwise direction and the shaft rear part 240 may extend along the second lengthwise direction, there is a lower risk that the couplers 210 will obstruct one another, when more than one of such surgical instruments are used in surgery. Therefore, the surgical instruments and robotic surgery can be applied even to microsurgery and SPA surgery procedures. In particular, if a surgical instrument structured as above, i.e. folded and extending along a first direction and a second direction, is joined with a robot arm, a greater level of freedom can be provided in terms of the direction in which the robot arm is installed and the direction in which the surgical instrument is extended, etc. The user may thus utilize the surgical instruments in a manner similar to using one's own arms, making it possible to conceive new surgical techniques.

In this embodiment, a wire can be used for connecting the driving wheel with the effector 250. That is, when the driving wheel is rotated, the movements of the effector 250 can be controlled as the wire joined with the driving wheel is contracted or relaxed. The number of driving wheels can be determined in correspondence with the structure for controlling the movements of the effector 250 and the number of wires used.

The effector 250 may be joined to the other end of the shaft rear part 240 and may be inserted into the body of the surgery patient. The effector 250 is the member that engages the surgical site during actual surgery. The effector 250 of the surgical instrument may include a pair of jaws, which may perform a gripping or cutting movement. Also, the effector 250 can be formed such that the whole of the effector 250 is able to rotate in linkage with the rotation of the shaft rear part 240.

In this case, the driving wheels of the driving part can be pulley-joined with the pair of jaws. Various methods can be used for joining the driving wheels with the pair of jaws, such as joining a set of wires to each of the jaws or joining a set of wires to the pair of jaws, for example. Referring to the latter case, as the driving wheels are rotated, the driving forces may be transferred by way of the wires, so that the pair of jaws may perform a gripping or cutting movement. In moving the pair of jaws using a set of pulley-wires, the pair of jaws may be connected by gears, etc., and the pulley-wires can be joined to one of the pair of jaws or to a portion where the pair of jaws are joined, to transfer the driving forces. Of course, various other mechanisms can be applied in which a set of pulleys are used that enable the pair of jaws to perform a gripping movement.

The shaft can be made to rotate about the first lengthwise direction, in which the shaft front part 220 is extended. In this case, the whole of the shaft rear part 240 can also rotate in correspondence with the rotation of the shaft front part 220, while extending in the second lengthwise direction.

Also, according to another embodiment, the shaft front part 220 and the shaft rear part 240 can be joined to each other in such a way that the shaft rear part 240 is rotatable at the first bending part about an axis following the second lengthwise direction described above. For example, the shaft front part 220 and the shaft rear part 240 can be bearing-joined with each other. Here, a bearing-joint refers to a joint that enables smooth rotational motion by reducing friction between the shaft front part 220 and the shaft rear part 240.

To enable this rotation of the shaft rear part 240, a separate wire can be used with one end joined with the driving wheel and the other end joined with the shaft rear part 240. Various methods can be used by which this wire joins the shaft rear part 240, such as winding the wire around the shaft rear part 240, or affixing the wire to a certain point on the shaft rear part 240, for example. Of course, various other mechanisms for rotating the shaft rear part 240 using a wire can be applied to this embodiment.

As described above, the shaft front part 220 and the shaft rear part 240 may be joined in such a way that the shaft rear part 240 is able to rotate about an axis following the second lengthwise direction. In this specification, this joining method will be referred to as a “bearing-joint.” Here, a bearing-joint not only includes linking structures such as a ball bearing, roller bearing, and plate bearing, but also encompasses various other linking structures, such as a screw-joint along an axis following the second lengthwise direction, and a linking structure that surrounds the perimeter of the shaft rear part 240 and uses a linking member that is held in an indentation formed in the perimeter. This bearing-joint may have a rotatable structure, to allow the shaft rear part 240 to rotate while extending in the second lengthwise direction, and obviously, various other bearing-joints can be applied to this embodiment.

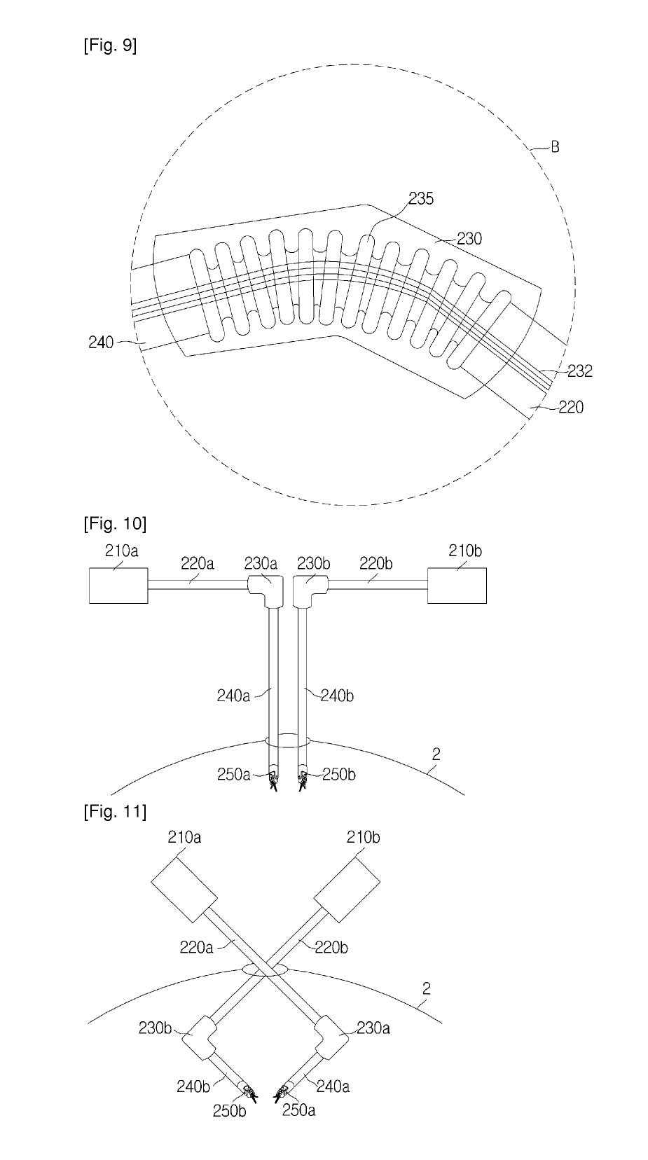

FIG. 9 is a perspective view illustrating the bending part of a flexible surgical instrument according to an embodiment of the present invention. In FIG. 9, which is a magnified view of area B, there are illustrated a shaft front part 220, a cover part 230, first wires 232, a first bending part 235, and a shaft rear part 240.

A first wire 232 may join the driving wheel with the effector 250 such that the effector 250 can be moved by the operation of the driving wheel. The first wire 232 may be pulley-joined to the driving wheel, to be moved in one direction in correspondence with the rotation of the driving wheel, where the effector 250 may perform a particular action in correspondence with this movement. For joining the first wire 232 to the driving wheel and the effector 250, a hole can be formed in the first bending part 235 along the direction in which the first bending part 235 is extended, and the first wire 232 can extend through this hole.

According to another embodiment, the first wire 232 may have one end joined to a portion of the driving wheel and the other end joined to a portion of the effector 250. The rotational movement of the driving wheel may cause the first wire 232 to undergo a contracting or relaxing motion, and in correspondence to this motion, the effector 250 may perform a particular operation, such as a gripping operation or a cutting operation.

The first bending part 235 may be formed as a bendable structure or from a bendable material. For example, the first bending part 235 can be a structure that includes a multiple number of separate articulated parts and is bent when a certain amount of force is applied in a particular direction. Also, the first bending part 235 can be made from a material high in plasticity, such as a synthetic resin tube. Furthermore, the whole shaft can be made as a bendable structure or made from a bendable material, with a cover part 230 used for encasing the bent location and maintaining its shape.

FIG. 10 and FIG. 11 are diagrams illustrating the uses of flexible surgical instruments according to embodiments of the present invention. Illustrated in FIG. 10 and FIG. 11 are couplers 210a, 210b, shaft front parts 220a, 220b, cover parts 230a, 230b, shaft rear parts 240a, 240b, effectors 250a, 250b, and a surgery patient 2.

In the example shown in FIG. 10, two flexible surgical instruments according to this embodiment are inserted through one hole formed in the skin of the surgery patient 2, and the cover parts 230a, 230b are not inserted through the hole but are positioned outside the skin of the surgery patient 2. As the shaft front parts 220a, 220b, which join with the shaft rear parts 240a, 240b, may extend in different directions, the couplers 210a, 210b may not obstruct each other. Here, the lengths of the shaft rear parts 240a, 240b can be greater than the lengths of the shaft front parts 220a, 220b. A laparoscope can additionally be inserted when conducting laparoscopic surgery, and a microscope can additionally be inserted when conducting microsurgery, but vision systems such as the laparoscope or microscope have been omitted from the drawings for more convenience.

In the example shown in FIG. 11, two flexible surgical instruments according to this embodiment are inserted through one hole formed in the skin of the surgery patient 2, and the cover parts 230a, 230b are inserted through the hole, to be positioned inside the skin of the surgery patient 2. To conduct surgery more smoothly for this situation, the lengths of the shaft rear parts 240a, 240b can be shorter than the lengths of the shaft front parts 220a, 220b. For example, when conducting SPA surgery, the effectors 250a, 250b can be moved towards the surgical site more easily and more efficiently, if the second shaft rear parts 240a, 240b are shorter than the shaft front parts 220a, 220b as in FIG. 11.

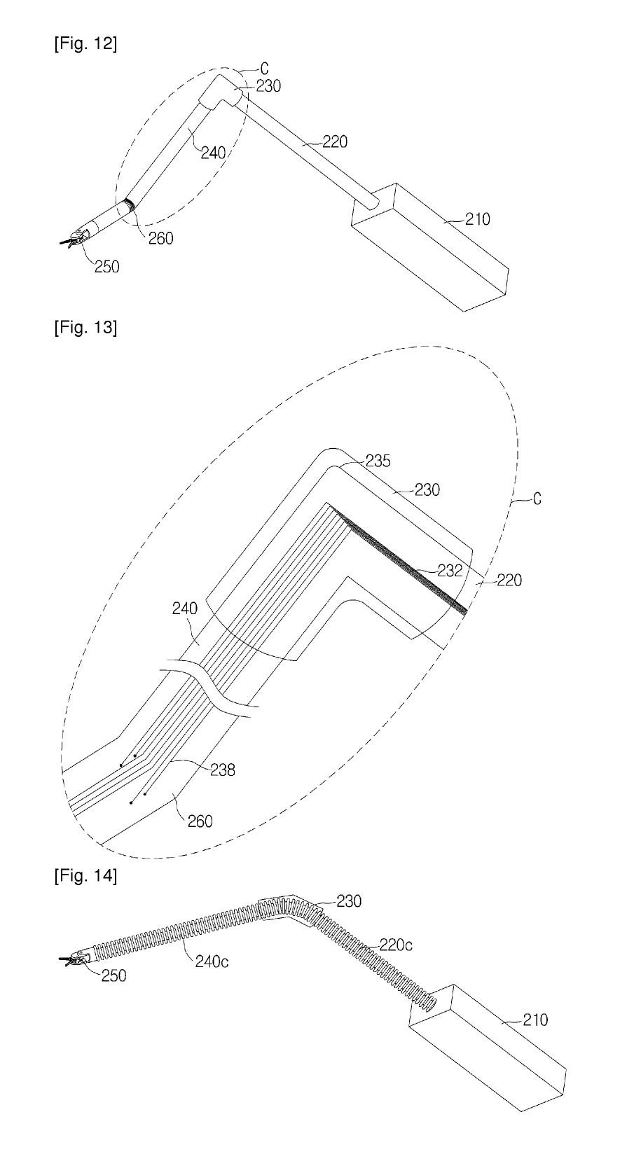

FIG. 12 is a perspective view of a flexible surgical instrument according to another embodiment of the present invention. Illustrated in FIG. 12 are a coupler 210, a shaft front part 220, a cover part 230, a shaft rear part 240, an effector 250, and a second bending part 260. The following descriptions will focus mainly on the differences from the previously described embodiment.

The second bending part 260 may be positioned between the shaft rear part 240 and the effector 250 and may have a bendable structure. Here, to state that the second bending part 260 may be positioned between the shaft rear part 240 and the effector 250 is intended to encompass not only those cases where the second bending part 260, i.e. a bendable member, is formed over all of the length between the shaft rear part 240 and the effector 250, but also those cases where the second bending part 260 is included at one end of the shaft rear part 240 and the effector 250 is joined to the far end after a particular length extending from the second bending part 260, as illustrated in the drawing.

The second bending part 260 may form a particular angle with the second lengthwise direction in which the shaft rear part 240 is extended, and may be formed as a bendable structure or from a bendable material. Similar to the first bending part 235 described above, the second bending part 260 can be a structure that includes a multiple number of separate articulated parts and is bent when a certain amount of force is applied in a particular direction or can be made from a material high in plasticity, such as a synthetic resin tube.

The second bending part 260 may be controlled by the operation of driving wheels, and for this purpose, the second bending part 260 and the driving wheels can be connected by wires. Referring to FIG. 13, which is a magnified view of area C, second wires 238 may connect the driving wheels with the second bending part 260, whereby the movement of the second bending part 260 can be controlled by the manipulation of the driving wheels. The second wires 238 may each have one end attached to one of four portions, respectively, within the second bending part 260, for example in intervals of 90 degrees. The other ends of the second wires 238 may be joined to the driving wheels, and the rotational movements of the driving wheels may contract or relax the second wires 238 to adjust the tensional forces applied, so that the angle and direction in which the second bending part 260 is bent may be determined accordingly. To implement such movements, additional driving wheels can be provided for manipulating the second bending part 260. Of course, various other mechanisms for bending the second bending part 260 using the second wires 238 can be applied to this embodiment.

Providing the surgical instrument with the second bending part 260 can increase the degree of freedom in controlling movements, so that the surgery may be conducted with greater convenience. That is, if second bending parts 260 are included in the examples of FIG. 10 and FIG. 11, the effectors 250a, 250b may be positioned in the surgical site more conveniently and more efficiently.

FIG. 14 is a perspective view of a flexible surgical instrument according to another embodiment of the present invention. Illustrated in FIG. 14 are a coupler 210, a shaft front part 220c, a cover part 230, a shaft rear part 240c, and an effector 250. The following descriptions will focus mainly on the differences from the previously described embodiments.

A feature of this embodiment is that the whole shaft 220c, 240c is implemented in a flexible form. This may be achieved by forming the shaft 220c, 240c from a material which is itself bendable or by forming the shaft 220c, 240c as a bendable structure. The shaft 220c, 240c may bend when the user applies an amount of force greater than a threshold value, and after it is bent, may bend or unbend to another angle when a force greater than the threshold value is applied again. Here, the threshold amount of force can be set such that the flexible surgical instrument according to this embodiment is not randomly unbent or bent in another direction during use in surgery.

For example, the shaft 220c, 240c can be a corrugated tube capable of bending. Here, the corrugated tube can be made from a common synthetic resin or metal, while a laminate made of synthetic resin may be applied on the exterior.

The cover part 230 may serve to hold the bent portion after the user bends the shaft 220c, 240c and to maintain the bent angle of the shaft 220c, 240c. To this end, the cover part 230 can have a form that remains secured in an angled state. In this case, several types of cover parts 230 can be prepared, each bent at a different angle. After determining the angle by which the shaft 220c, 240c is to be bent when conducting surgery, the user may select the cover part 230 corresponding to this angle and position the cover part 230 at the bent portion of the shaft 220c, 240c, so that the shaft 220c, 240c may maintain its bent angle.

According to another embodiment, the cover part 230 itself can also be made flexible. In this case, the cover part 230 can be stiffer and more resistant to bending, compared to the shaft 220c, 240c. That is, in order that the cover part 230 may serve to maintain the bent state of the shaft 220c, 240c, the threshold force described above can be greater for the cover part 230 compared to the shaft 220c, 240c.

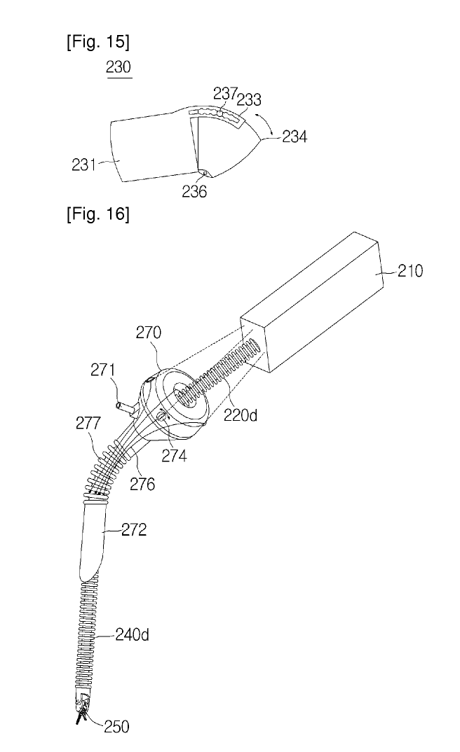

FIG. 15 is a drawing illustrating a cover part of a flexible surgical instrument according to an embodiment of the present invention. Illustrated in FIG. 15 are a first cover part 231, a stopper 233, a second cover part 234, a rotational axis 236, and a fastening part 237.

According to this embodiment, a cover part 230 is provided which can be varied in its bending angle while maintaining a rigid state. This cover part 230 can be adjusted in correspondence to the angle of the shaft 220, 240 in a flexible surgical instrument according to this embodiment.

The cover part 230 may have a first cover part 231 extending towards the shaft front part 220c and a second cover part 234 extending towards the shaft rear part 240c. The first cover part 231 and second cover part 234 can be hinge-joined about a rotational axis 236, to be capable of rotational movement. A stopper 233 can, in linkage with a fastening part 237, adjust the joint angle between the first cover part 231 and the second cover part 234. That is, the stopper 233 can be joined to the first cover part 231 and can include a multiple number of detent curbs formed along a particular circumference centering about the rotational axis 236. The fastening part 237 can be joined to one of the detent curbs, to secure the second cover part 234 in a rotated position about the rotational axis 236. For this purpose, the fastening part 237 can be formed as a protrusion in a particular position of the second cover part 234.

Using this structure, the cover part 230 can be secured while maintaining a particular angle between the first cover part 231 and the second cover part 234. Also, the fastening part 237 can be screw-joined with the second cover part 234. When the second cover part 234 is to be rotated in relation to the first cover part 231, for example, the screw-joint of the fastening part 237 can be unscrewed, to rotate the second cover part 234, and then tightened again, to secure the second cover part 234.

While the description above has been set forth with reference to an example in which the stopper 233 is formed in the first cover part 231 and the fastening part 237 is formed on the second cover part 234, it is obvious that the stopper 233 can be formed in the second cover part 234 and the fastening part 237 can be formed on the first cover part 231.

FIG. 16 is a drawing illustrating a linking structure between a flexible surgical instrument and a medical trocar according to an embodiment of the present invention. Illustrated in FIG. 16 are a coupler 210, a shaft front part 220d, a medical trocar, a shaft rear part 240d, and an effector 250. The medical trocar can include a trocar housing 270, a vent tube 271, a cannula 272, drive valves 274, third wires 276, and a third bending part 277. The following descriptions will focus mainly on the differences from the previously described embodiments.

A medical trocar is a medical tool typically used to access the abdominal cavity. During surgery, a medical tool such as a laparoscope and a surgical instrument may be inserted using a medical trocar. In order to insert a flexible surgical instrument such as those described above, a medical trocar according to this embodiment can be made with a flexible form.

The cannula 272, which is to be inserted through the skin of the patient, can include a third bending part 277 that can be bent at a particular position. The third bending part 277 can be implemented by a particular material or structure as described above. Also, according to another embodiment, the whole of the cannula 272 can have a flexible form. Since this structure can be implemented in a manner similar to the shaft of the flexible surgical instrument described above, details on this matter will be omitted. As described above, the threshold force required for bending the cannula 272 can be greater than the threshold force for bending the flexible surgical instrument, whereby the cannula 272 can maintain the bent angle of the flexible surgical instrument.

Gases within the body can be exhausted to a pre-arranged location (e.g. a vacuum suction tube or an air vent of the operating room) through the cannula 272, as well as a vent tube 271 and a vacuum connection tube (not shown), which may be prepared additionally.

The drive valves 274 can be provided to adjust the angle by which the cannula 272 is bent. That is, the drive valves 274 and certain points on the cannula 272 may be connected by third wires 276, where the rotation or movement of the drive valves 274 may adjust the tensional forces applied on the third wires 276 and thus bend the cannula 272 in a particular direction.

That is, the third wires 276 may each have one end attached to one of four portions, respectively, within the cannula 272, for example in intervals of 90 degrees. The other ends of the third wires 276 may be joined to the drive valve 274, and the rotational movements of the drive valve 274 may contract or relax the third wires 276 to adjust the tensional forces applied, so that the angle and direction in which the cannula 272 is bent may be determined accordingly. Of course, various other mechanisms for bending the cannula 272 using the third wires 276 can be applied to this embodiment.

According to another embodiment, the drive valves 274 can be connected by wires to driving wheels of the coupler 210. That is, the manipulation of the actuator of the robot arm can move the driving wheels of the coupler 210, and the drive valves 274 can be controlled correspondingly to bend the cannula 272 in a particular direction. This embodiment provides the advantage that a user may bend the medical trocar at will using a master robot.

While FIG. 16 illustrates an example in which the medical trocar includes one passageway through which to insert a medical tool, the present invention is not thus limited. A medical trocar according to another embodiment can include multiple passageways, for example with several holes perforated for single port surgery.

Other details related to the flexible surgical instrument according to an embodiment of the present invention or related to the surgical robot which the instrument may operate in linkage with, including, for example, detailed mechanical designs, common platform technology, such as the embedded system, O/S, etc., interface standardization technology, such as the communication protocol, I/O interface, etc., and component standardization technology, such as for actuators, batteries, cameras, sensors, etc., are obvious to those of ordinary skill in the field of art to which the present invention belongs and thus will be omitted here.

While the flexible surgical instrument according to an embodiment of the present invention has been described above with reference to certain examples regarding the number and functions of the shafts, the present invention is not thus limited. Other compositions, in which the shaft is divided into smaller segments, or in which the operation method does not utilize wires, for example, can be encompassed by the scope of claims of the present invention if the overall actions and effects are substantially the same.

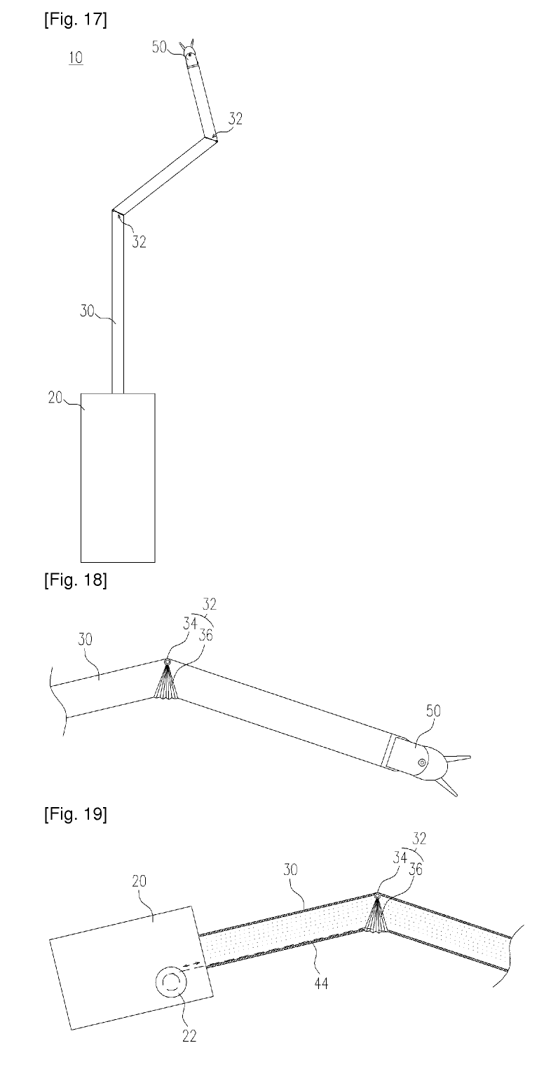

FIG. 17 is a diagram schematically illustrating a surgical instrument according to an embodiment of the present invention, and FIG. 18 is a magnified view of the elbow portion of a surgical instrument according to an embodiment of the present invention. Illustrated in FIG. 17 and FIG. 18 are an instrument 10, a driving part 20, a shaft 30, elbows 32, a hinge axis 34, an expandable part 36, and an effector 50.

A feature of this embodiment is that an elbow structure is applied to the middle of the shaft 30 in the surgical instrument, so that the shaft 30 may be curved in the middle. Thus, when the far end of the shaft 30, i.e. the effector 50, is inserted into the body during a surgical procedure, a surgeon may manipulate the surgical instrument just as if the surgeon's own arms are moved inside the body.

An instrument 10 according to this embodiment can be composed mainly of a driving part 20, a shaft 30 extending in one direction from the driving part 20, and an effector 50 joined to the far end of the shaft 30. In the case of a robotic surgical instrument, the driving part 20 may be the part that is mounted on a surgical robot to receive driving forces transferred from the surgical robot, and in the case of a manually operated instrument, the driving part 20 may be the part that is held and manipulated by the user to receive its driving forces directly from the hands of the user.

Onto this driving part 20, a driving wheel or driver can be installed which engages an actuator of the robot, or a handgrip such as a wheel, lever, switch, etc., can be installed which may be held by the user. When a driving force is transferred from the robot, or when the user manually manipulates the driving part 20, the effector 50 may accordingly move in a gripping, rotating, tilting movement, etc., to implement a maneuver required for surgery.

In other words, the driving part 20 according to this embodiment can be configured to couple onto a surgical robot arm and be manipulated by driving forces transferred from the robot, in the case of a robotic surgical instrument, and can be configured to be manually manipulated by the user, in the case of a manually operated instrument.

The shaft 30 can be shaped as a straight line extending in one direction, and by using a tube member having a typical cylindrical shape, etc., the shaft 30 can hold the pulley-wires that connect the driving part 20 with various portions of the effector 50 to transfer the driving forces from the driving part 20 to the effector 50. Thus, when portions of the driving part 20 are manipulated, the respective portions of the effector 50 connected by pulley-wires may be moved.

As illustrated in FIG. 17, the shaft 30 of an instrument 10 according to this embodiment can have elbows 32 formed in the middle, enabling the shaft 30 to curve at the elbows 32. An elbow 32 may serve as an articulation at which the straight shaft 30 may bend by a particular angle. The function of the elbow 32 can be implemented by forming the elbow 32 portion, or the entire shaft 30, in the shape of a corrugated tube or bellows.

As illustrated in FIG. 18, an elbow 32 according to this embodiment can be composed with a hinge axis 34 formed on one side and an expandable structure on the other, when looking at the cross section of the shaft 30. In this way, the shaft 30 may be curved at the elbow 32, to be more specific, at the hinge axis 34, in a direction that contracts the expandable part 36. Thus, for a shaft 30 according to this embodiment, the direction and the degree in which the shaft 30 is curved can be determined by the structure of the elbows 32 formed in the middle.

The expandable part 36 is a component that enables to shaft 30 to bend or unbend while maintaining its shape. The expandable part 36 can be shaped as a corrugated tube or bellows, or can be made from a flexible material.

Furthermore, the expandable part 36 can include an elastic body that applies an elastic force in a direction that expands the expandable part. That is, an elastic body such as a spring, etc., can be included in the expandable part, while a stopper, etc., can be formed in the hinge axis to prevent the expandable part from expanding excessively. Then, the shaft may normally remain in a straight, unbent state, but when it is pulled using a wire, etc., the expandable part may contract and the shaft may bend at the elbow, and when the tensional force on the wire is removed, the shaft may return to its unbent state due to the restoring force of the elastic body.

Alternatively, the expandable part 36 can include an elastic body such as a spring, etc., that applies an elastic force in a direction that contracts the expandable part. Then, the shaft may normally (when there is no force applied) remain in a bent state, but when a force is applied using a wire, etc., the expandable part may expand and the shaft may be unbent into a straight form, and when the external force is removed, the shaft may return to its bent state due to the restoring force of the elastic body. Such configurations can be used to improve safety during surgical procedures.

A description will now be provided as follows on the operation of an instrument 10 according to this embodiment, using an example that includes the elbow structure illustrated in FIG. 18.

FIG. 19 is a diagram illustrating the operation of a surgical instrument according to an embodiment of the present invention. Illustrated in FIG. 19 are a driving part 20, a driver 22, a shaft 30, an elbow 32, a hinge axis 34, an expandable part 36, and a wire 44.

A shaft 30 in which an elbow 32 is formed according to this embodiment can be operated by the tension of the wire 44. That is, a wire 44 can be connected near the elbow 32 and connected to the driving part 20, whereby the shaft 30 can be made to fold at the elbow 32 by manipulating the driving part 20 to apply a tensional force on the wire 44.

Referring to the portion of the driving part 20 where the wire 44 is connected as the driver 22, the shaft 30 of an instrument 10 according to this embodiment may be curved at the elbow 32 according to the manipulation of the driver 22. The driving part 20 can be equipped with other drivers 22 for operating the effector 50, and these other drivers 22 can be connected with other wires, which connect to the effector 50. Details on the structure, function, operating method, etc., of the drivers 22 and wires for operating the effector 50 will be omitted here, and in the descriptions that follow, the terms “driver” and “wire” will refer to the driver 22 and wire 44 for curving the shaft 30, respectively, unless otherwise stated.

As already described above, a shaft 30 according to this embodiment can be made from a tube-shaped member having a typical cylindrical shape, etc. In this case, the wire 44 may be held within the shaft 30 and extend along the lengthwise direction of the shaft 30 to be connected to a particular position near the elbow 32.

As illustrated in FIG. 19, a shaft 30 according to this embodiment can include a multiple number of elbows 32. For example, if a shaft 30 according to this embodiment were to be compared to a human arm, the elbows 32 illustrated in FIG. 19 can be regarded as corresponding to the elbow and wrist joints.

In certain cases where the effector 50 joined to the end of the shaft 30 is to be drawn close to or away from the driving part 20 by curving the shaft 30, it is possible to form the structure of the elbows 32 such that the shaft 30 is folded in a zigzag shape, i.e. with each elbow curving the shaft in opposite directions. Thus, just as a person is able to move one's hand closer to or further from the shoulder according to the movement of the elbow and wrist joints, the effector 50 can be moved closer to or further from the driving part 20 by bending or unbending the shaft 30 at each of the elbows 32.

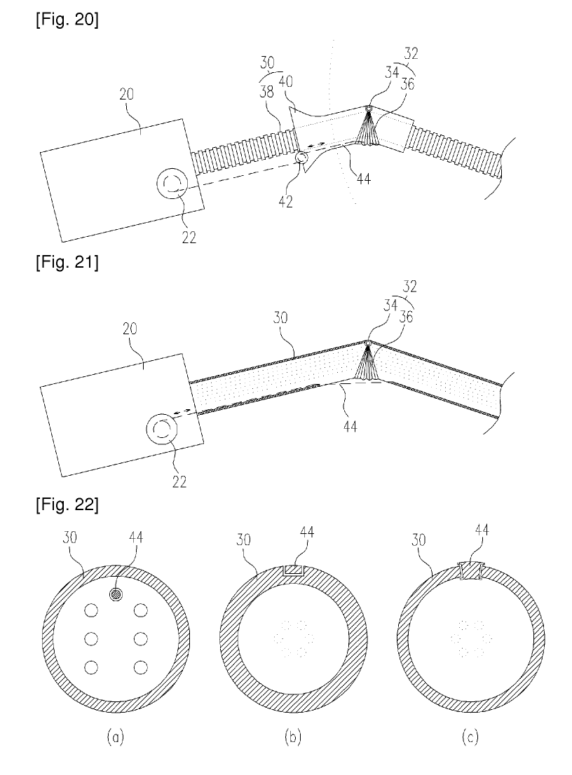

FIG. 20 is a diagram illustrating the operation of a surgical instrument according to another embodiment of the present invention. Illustrated in FIG. 20 are a driving part 20, a driver 22, a shaft 30, an elbow 32, a hinge axis 34, an expandable part 36, a core 38, a guide member 40, a driving wheel 42, and a wire 44.

This embodiment relates to forming the shaft 30 as a dual structure, i.e. including an inner core 38 that serves as a channel for holding the wire 44 and a guide member 40 that surrounds the core 38. The core 38 can be made from a flexible material, to be capable of bending freely, and the rigid guide member 40 can surround the perimeter of the core 38, with an elbow 32 such as that described above formed in the middle of the guide member 40. Thus, the core 38 can be curved, i.e. the shaft 30 can be curved, by curving the guide member 40.

In this case, the core 38 may be made from a material and/or structure, such as of a corrugated tube, etc., which is flexible but does not change shape unless an external force is applied. The core 38 may then maintain a certain shape (e.g. a straight line), until the guide member 40 is curved at the elbow 32, when the core 38 may change to a curved shape, after which the core 38 may remain in this changed state.

A guide member 40 according to this embodiment can also be used as a surgical trocar. In this case, the guide member 40 (trocar) may first be inserted into the surgical site, and then the core 38 of the instrument 10 may be inserted through the trocar, so that the core 38 inserted through the guide member 40 (trocar) may, as a whole, serve as the shaft 30. If the shaft 30 is to be curved to a particular angle, the guide member 40 may be bent at the elbow 32 formed in the guide member 40, causing the core 38 to change shape accordingly, and consequently causing the shaft 30 to curve.

For curving the guide member 40, it is possible to connect a wire 44 to the vicinity of the elbow 32 of the guide member 40 and apply a tensional force on the wire 44 to curve the guide member 40 at the elbow 32, similar to the previously described embodiments. Moreover, the guide member 40 can be made to curve at the elbow 32 due to the tension on the wire 44, by including a driver 22 in the driving part 20, connecting the wire 44 to the driver 22, and manipulating the driver 22.

It is also possible to join a separate driving wheel 42 to the guide member 40 and connect the wire 44 to the driving wheel 42, so that the guide member 40 may be curved when a tensional force is applied on the wire 44 according to the manipulation of the driving wheel 42. In cases where the guide member 40 is used as a trocar as described above, the instrument 10 may be inserted through the guide member 40, and afterwards the trocar, i.e. the guide member 40, can be bent by a particular angle by manipulating the driving wheel 42 joined to the guide member 40.

The manipulation for bending the guide member 40 after joining a separate driving wheel 42 can be performed manually, or the driving wheel 42 can be connected to the driver 22 included in the driving part 20, so that the driving wheel 42 may be manipulated in linkage with a manipulation on the driver 22. Of course, various mechanical connection methods, such as pulley-wires and links, etc., can be applied for linking the operation of the driving wheel 42 to that of the driver 22.

In such cases where a driving wheel 42 is joined to the guide member 40 and a driver 22 is included in the driving part 20, the driving wheel 42 can be made to operate in linkage with the manipulation of the driver 22 by connecting the driving wheel 42 with the driver 22 during or after the process of inserting the core 38 of the instrument 10 through the guide member 40.

FIG. 21 is a diagram illustrating the operation of a surgical instrument according to another embodiment of the present invention. Illustrated in FIG. 21 are a driving part 20, a driver 22, a shaft 30, an elbow 32, a hinge axis 34, an expandable part 36, and a wire 44.

The wire 44 used for applying a tensional force to curve the shaft 30 at the elbow 32 can be held within the shaft 30 as described above, but can also be exposed at the surface of the shaft 30, or configured to be pulled out of the shaft 30.

That is, if the wire 44 connecting the driver 22 with the elbow 32 is held inside the shaft 30, the process of curving the shaft 30 by applying tension on the wire 44 can entail an amount of friction generated between the wire 44 and the bent portion within the shaft 30. This may create a risk of damage to the wire 44 and/or the shaft 30 as well as a risk of malfunctioning in the curving operation.

To prevent such risks, a different material can be used for a portion of the shaft 30, or a separate bearing member, etc., can be used, to minimize friction between the wire 44 and the bent portion of the shaft 30. Alternatively, a portion of the can be uncovered, as illustrated in FIG. 21, so that the wire 44 may be pulled out of the shaft 30 when a tensional force is applied on the wire 44.

For example, a slit can be perforated in a portion of the shaft 30, and the shaft 30 can be installed in such a way that the wire 44 can be exposed through the slit at the surface of the shaft 30. Then, as the shaft 30 is curved, the wire 44 can be pulled out of the shaft 30 in correspondence to the shortest distance between the elbow 32 and the driving part 20, so that unnecessary friction between the wire 44 and the shaft 30 can be minimized, and the tensional force can be effectively delivered through the wire 44.

FIG. 22 is a diagram illustrating possible cross sections for the shaft of a surgical instrument according to an embodiment of the present invention. FIG. 22 shows illustrations of shafts 30 and wires 44.

The following relates to examples of cross sections for the shaft 30, in cases where the wire 44 is held inside the shaft 30 or exposed at the surface of the shaft 30, as mentioned with regard to the previously described embodiment.

Drawing (a) of FIG. 22 illustrates a shaft 30 having a circular cross section, where the channels for holding a multiple number of wires are perforated separately. Not only the wire 44 according to this embodiment but also other wires for operating the effector 50 can be held within the perforated channels. This allows the wires to effectively transfer the tensional forces generated according to the manipulation of the driving part 20 without interfering or causing friction with one another within the shaft 30.

Drawing (b) of FIG. 22 illustrates a shaft 30 having a circular cross section, where the wires for operating the effector 50 are held inside, and the wire 44 according to this embodiment is exposed at the surface of the shaft 30. In order to provide a smooth surface for the shaft 30, without having the wire 44 protrude out from the surface of the shaft 30, a portion of the exterior of the shaft 30 can be recessed to form a trough, such as that illustrated in drawing (b) of FIG. 22, and the wire 44 can be installed with a cross section corresponding with that of the trough.

Drawing (c) of FIG. 22 illustrates the cross section of a shaft 30 that is formed as a partially opened cylinder, where the wires for operating the effector 50 are held inside, and the wire 44 according to this embodiment is installed to cover the open portion of the shaft 30. That is, the wire 44 may form a portion of the perimeter of the shaft 30, so that normally, the wire 44 may close off the space within the shaft 30.

For the examples shown in drawings (b) and (c) of FIG. 22, the wire 44 may be pulled out of the shaft 30 when a tensional force is applied on the wire 44 to curve the shaft 30, as described above with reference to FIG. 21, so that unnecessary friction between the wire 44 and the shaft 30 can be minimized, and the tensional force can be effectively delivered through the wire 44.

Although it is not illustrated in the drawings, it is also conceivable, instead of using the tube-shaped shaft 30, to have the wire 44 according to this embodiment and the wires for operating the effector 50 combine together and form a cross section for a shaft 30. In this case, the wire 44 according to this embodiment can be exposed at the surface of the shaft 30 and may be naturally pulled out of the shaft 30 as the shaft 30 is curved.

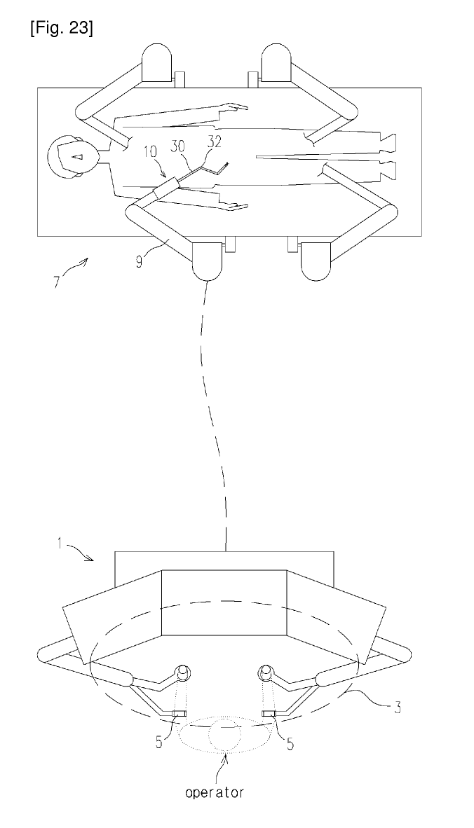

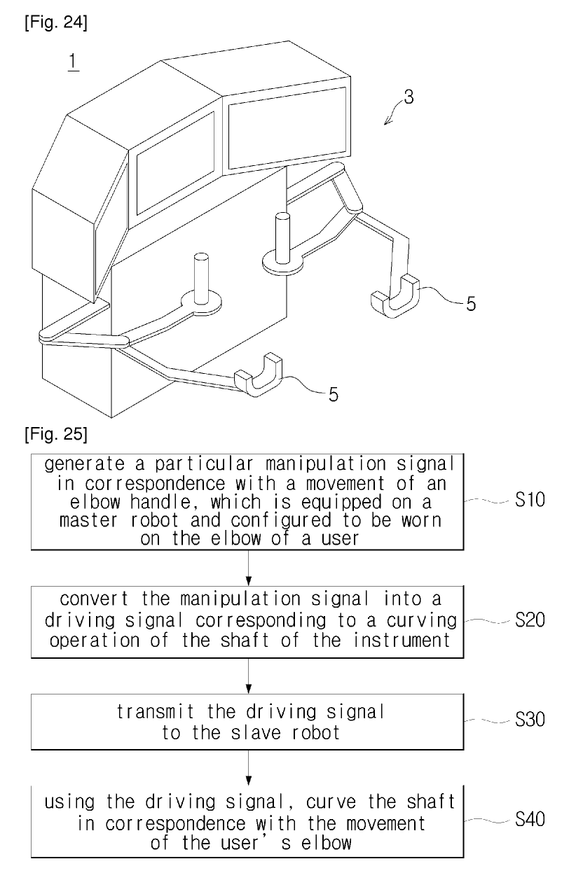

FIG. 23 is a diagram illustrating the composition of a surgical robot according to an embodiment of the present invention, and FIG. 24 is a perspective view of a master interface for a surgical robot according to an embodiment of the present invention. Illustrated in FIG. 23 and FIG. 24 are a master robot 1, an interface 3, elbow handles 5, a slave robot 7, robot arms 9, an instrument 10, a shaft 30, and an elbow 32.

This embodiment relates to a surgical robot that may be driven after mounting an instrument 10 described above, as well as to a master interface for the surgical robot. That is, as a means to make manipulations for curving the shaft 30 of the instrument 10, the master interface 3 may be equipped with handles dedicated to inputting these manipulations. A particular signal generated in accordance with a manipulation on the dedicated handles may be transferred to the slave robot 7 to correspond with a curving action of the shaft 30. In the descriptions that follow, these handles dedicated to this purpose will be referred to as “elbow handles.”

A surgical robot according to this embodiment may include a master robot 1 and a slave robot 7. An interface 3 that enables a user to make manipulations may be installed in the master robot 1, and when a manipulation is inputted, by way of various handles, levers, buttons, clutches, etc., equipped on the interface 3, a corresponding signal may be transmitted to the slave robot 7 and the slave robot 7 may be operated.

The slave robot 7 can be equipped with one or more robot arms 9, to which a surgical instrument 10 may be mounted. Each robot arm 9, as well as the instrument 10 mounted on the robot arm 9, may be driven according to a signal transmitted from the master robot 1 to conduct surgery.

On a master interface 3 according to this embodiment, a separate elbow handle 5 can be installed for generating a particular manipulation signal. As already described above, an instrument 10 according to this embodiment can include an elbow 32 formed in the shaft 30, and the shaft 30 can curve at the elbow 32, so the manipulation signal generated according to the manipulation of the elbow handle 5 may be transmitted to the slave robot 7 and used in curving the shaft 30 of the instrument 10.

As described above for the previously disclosed embodiments, a feature of an instrument 10 according to this embodiment is that the shaft 30 can be curved, in a manner analogous to an elbow joint. As such, the elbow handle 5 can be installed in a shape and structure that allows the elbow handle 5 to be worn on the elbow of the user. Then, the user may wear the elbow handle 5 on the elbow and move the elbow handle 5, causing the shaft 30 to operate in correspondence with the movement of the user's elbow.

For this purpose, an elbow handle 5 according to this embodiment can be formed as a

U-shaped armrest into which the elbow portion of the user may be inserted. After inserting the elbow portion into this elbow handle 5, the user may manipulate the shaft 30 of the instrument 10 just as if the user were moving one's own arm, and the user may manipulate the robot more intuitively.

FIG. 25 is a flowchart illustrating a method of driving a surgical robot according to an embodiment of the present invention. This embodiment relates to a method of driving an instrument 10 mounted on a slave robot 7 by manipulating the master interface 3 described above.

That is, this embodiment provides a method of driving an instrument 10, which has a curvable shaft 30, and which is mounted on a slave robot 7, by manipulating a master robot 1 connected to the slave robot 7. First, the separate elbow handle 5 installed on the master interface 3 may be manipulated. The elbow handle 5 is a dedicated handle included in the master interface 3 that is configured to be worn on the elbow of a user. In correspondence with the movement of the elbow handle 5, a particular manipulation signal may be generated (S10).

The generated manipulation signal may be converted into a particular driving signal that corresponds to a curving operation of the shaft 30 (S20), and the converted driving signal may be transmitted to the slave robot 7 (S30), allowing the shaft 30 of the instrument 10 to operate in correspondence with the manipulation of the elbow handle 5. Thus, in an instrument 10 according to this embodiment, the shaft 30 may undergo a curving movement according to the movement of the elbow of the user manipulating the master interface 3 (S40). In this way, a user may intuitively manipulate the instrument 1 on a surgical robot according to this embodiment, just as if the user were moving his or her own arm.

The driving method for the surgical robot described above can also be implemented in the form of a computer program that is read and executed by a digital processing device, such as a microprocessor, etc., which may be either built into the robot itself or connected to the robot from an external source.

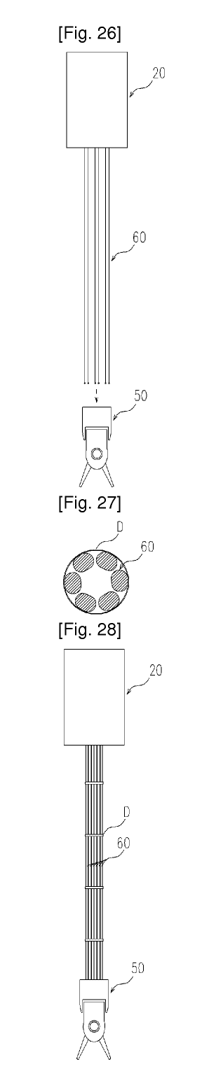

FIG. 26 is a diagram schematically illustrating a surgical instrument according to an embodiment of the present invention, and FIG. 27 is a lateral cross-sectional view of a set of rods according to an embodiment of the present invention. Illustrated in FIG. 26 and FIG. 27 are a driving part 20, rods 60, and an effector 50.

A feature of this embodiment is embodiment is that the effector 50 and the rods 60 of the surgical instrument are configured to be attachable and detachable in relation to each other, so that the rods 60 can be joined to the effector 50 after first inserting the separately detached effector 50 into the abdominal cavity and then invading the rods 60. Thus, the surgical instrument can be manipulated for surgery after making an incision that is small enough not to leave a scar.

The instrument according to this embodiment may be composed mainly of a driving part 20, a multiple number of rods 60 joined to the driving part 20, and an effector 50 detachably joined to the far end of the rods 60. The driving part 20 is a part that can be manually operated by a surgeon in the case of manual operation, and can be manipulated by driving forces transferred from the robot arm in the case of robotic surgery.

The rods 60 are components that may move along a particular lengthwise direction according to a manipulation on the driving part 20. For example, in cases where multiple driving wheels are mounted on the driving part 20 and the rods 60 are pulley-joined to the driving wheels, respectively, each rod 60 may serve to transfer a tensional force according to the rotation of the driving wheel. Alternatively, drivers that each perform a reciprocating movement along the lengthwise direction of the rods 60 can be mounted instead of the driving wheels, and the rods 60 can be joined to the drivers, in which case the rods 60 can be made to transfer forces along the lengthwise direction when the drivers are manipulated.

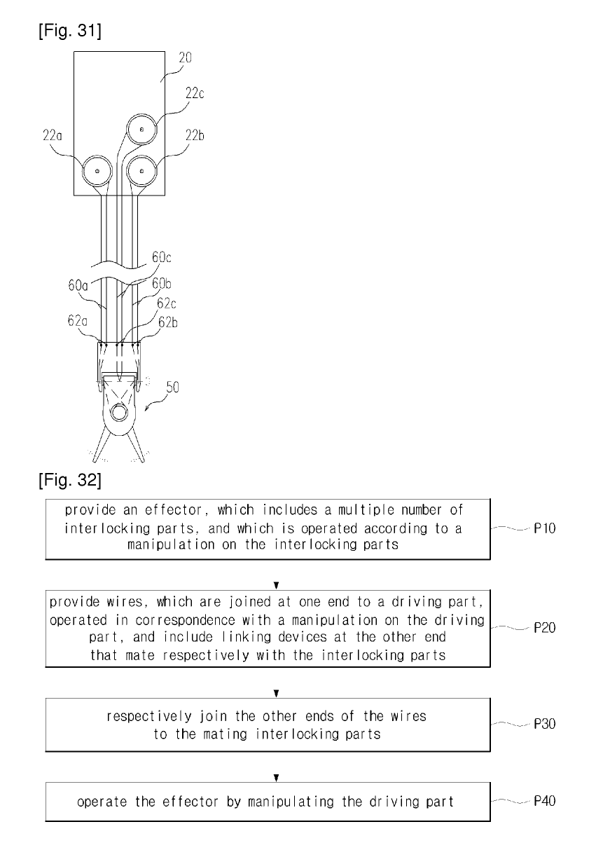

The effector 150 is the component that is actually inserted into the surgical site to perform a gripping or cutting movement, etc. The effector 50 according to this embodiment may be joined to the far end of the rods 60, configured such that the effector 50 can be separated from the rods 60 and the separated effector 50 can be reattached to the rods 60 as necessary. The linking structure between the rods 60 and the effector 50 will be described in further detail in the paragraphs describing FIG. 30.

When the effector 50 is thus joined to the far end of the rods 60, the rods 60 may transfer forces according to the manipulation of the driving part 20, causing the parts of the effector 50 to operate. As a result, the effector 50 may perform a gripping or cutting motion.

When using a surgical instrument according to this embodiment, the detached effector 50 may first be inserted into the surgical site, and then the rods 60 may invade the surgical site, after which the effector 50 may be joined to the far end of the rods 60 inside the surgical site and manipulated. Therefore, the separately detached effector 50 may be formed in a size that can be inserted into the surgical site, i.e. a size that allows the effector 50 to pass through a trocar inserted at the surgical site.

In this way, the effector 50 may be inserted through a trocar, which itself is inserted beforehand, and the instrument may invade the surgical site directly, where the effector 50 may afterwards be joined to the rods 60 inside the surgical site.

Furthermore, an instrument according to this embodiment can employ a set of rods 60 instead of a separate shaft member, as illustrated in FIG. 27, so that the diameter of the shaft (i.e. the set of rods 60) can be minimized. For example, if the diameter of the set of rods 60 is set to 2 mm or smaller, similar to the diameter of a syringe needle, then there is no need to suture the skin and there is no scar left behind in the skin after the rods 60 invade the skin of the surgical site, so that the laparoscopic surgery may be performed with a greater level of safety.

Thus, the multiple number of rods 60 according to this embodiment can form a set which itself may serve as the shaft, while one or more bands or rings (see “D” in FIG. 27) for binding the rods can be placed intermittently on the multiple number of rods.

FIG. 28 is a diagram schematically illustrating a surgical instrument according to another embodiment of the present invention. Illustrated in FIG. 28 are a driving part 20, rods 60, and an effector 50.

This embodiment provides an example of a surgical instrument that uses a set of multiple rods as a substitute for the shaft. The main composition of a driving part 20, a multiple number of rods 60 joined to the driving part 20, and an effector 50 detachably joined to the far end of the rods 60 is substantially the same as that of the previously described embodiment. As the functions, structures, and operating methods of the driving part 20 and the effector 50 are substantially the same as those of the previously described embodiment, details on this matter will be omitted here.

The multiple number of rods 60 according to this embodiment may form a set, to function as the “shaft” extending in a lengthwise direction. In other words, instead of using a separate shaft, a set of rods 60 can be used, with the several rods 60 gathered together, fastened together with bands or rings, etc., (see “D” in FIG. 28) in intervals along the middle to prevent the bundle of rods from being separated, or even twisted around one another to form a set. In this way, the diameter of the instrument can be minimized, and the surgical instrument can be used after making an incision in the surgical site that is small enough not to require suturing.

Of course, it is not imperative that the effector 50 and the rods 60 be connected to each other in implementing this embodiment, and a detachably attachable structure can be employed between the far end of the set of rods 60 and the effector 50, similar to the previously described embodiment.

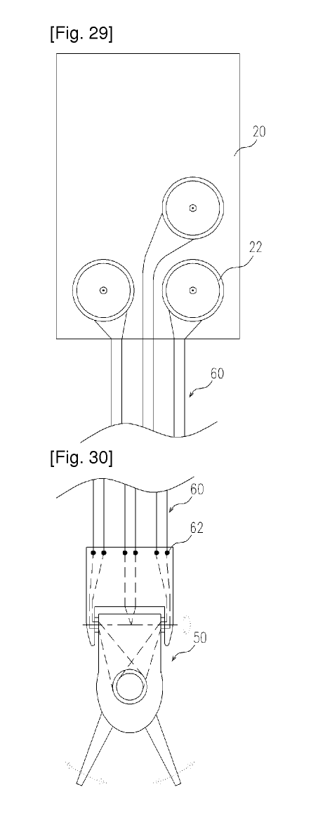

FIG. 29 is a diagram schematically illustrating the driving part of a surgical instrument according to an embodiment of the present invention. Illustrated in FIG. 29 are a driving part 20, drivers 22, and rods 60.

The embodiment shown in FIG. 29 illustrates an example in which there are a multiple number of wheel-shaped drivers 22 arranged in the driving part 20 [symbol] [symbol] driver 22, where a pair of rods 60 are joined to each of the drivers 22. As described above, various joining methods can be applied, such as pulley-joining the rods 60 to the drivers 22 and joining one end of each rod 60 to a portion of a driver 22.

When the wheel-shaped driver 22 is rotated, the rods 60 joined to the driver 22 may move along the lengthwise direction, thereby transferring the driving force to a part of the effector 50 joined to the other end of the rods 60.

However, FIG. 29 is an illustration of just one example. It is not imperative that the drivers 22 be limited to wheel-like shapes, neither is it imperative that a pair of rods 60 be joined to each driver 22. The composition of the drivers 22 and rods 60 can be implemented using various structures, for example with one rod 60 joined to one driver 22 that undergoes a reciprocating movement along the lengthwise direction of the rod 60.

FIG. 30 is a diagram schematically illustrating the effector of a surgical instrument according to an embodiment of the present invention. Illustrated in FIG. 30 are rods 60, an effector 50, and interlocking parts 62.

The effector 50 illustrated in FIG. 30 comprises three movable parts, namely, a pair of claws, each of which may rotate about a particular rotational axis, and a tilting axis, about which the whole of the forceps may perform a tilting movement. Thus, the effector 50 according to this embodiment may move with 3 degrees of freedom.

To enable each part of the effector 50 to move or rotate, each movable part may include two interlocking parts 62. Referring to two interlocking parts 62 equipped to rotate a claw, for example, pulling on one of the interlocking parts 62 may move the claw to open, while pulling on the other may move the claw to close. For the tilting axis, pulling on one of the two interlocking parts 62 corresponding to the tilting axis may tilt the set of claws in a plus (+) direction, while pulling the other may tilt the claws in a minus (−) direction.

If two interlocking parts 62 are thus provided for each movable part, then an effector 50 according to this embodiment that has 3 degrees of freedom may include a total of six interlocking parts 62. To these interlocking parts 62, the other ends of the rods 60 described above may be joined, respectively, so that the tensional forces transferred through the respective rods 60 may enable the parts of the effector 50 to operate according to the manipulations on the driving part 20.

However, FIG. 30 is an illustration of just one example. It is not imperative that the effector 50 operate with 3 degrees of freedom, neither is it imperative that a pair of interlocking parts 62 be included for each movable part of the effector 50. The composition of the effector 50 and interlocking parts 62 can be implemented in various ways, for example with one interlocking parts 62 included for one movable part and with the movable part configured to operate according to the pulling or pushing of the corresponding interlocking part 62.

FIG. 31 is a diagram illustrating the operation of a surgical instrument according to an embodiment of the present invention. Illustrated in FIG. 31 are a driving part 20, drivers 22a, 22b, 22c, rods 60a, 60b, 60c, an effector 50, and interlocking parts 62a, 62b, 62c.

FIG. 31 illustrates an example of an instrument formed by joining the driving part 20 with the effector 50 as described above. A description will now be provided as follows on the operation of an instrument according to this embodiment, with reference to FIG. 31.

As already described above, if the effector 50 according to this embodiment is a structure that operates with n degrees of freedom (n is a natural number), then a multiple number of interlocking parts 62a, 62b, 62c may be included in correspondence with the movable parts of the effector 50. Looking at the assembly shown in FIG. 30, for example, the effector 50 may operate with 3 degrees of freedom, and there may be two interlocking parts 62a, 62b, 62c corresponding with each movable part, resulting in a total of six interlocking parts 62a, 62b, 62c.

On the other hand, the driving part 20 according to this embodiment may also be included in multiple numbers in correspondence with the degree of freedom of the effector 50. In cases where the effector 50 operates with 3 degrees of freedom, as is the case shown in FIG. 30, the driving part 20 may correspondingly include three drivers 22a, 22b, 22c, so that the driving part 20 may be manipulated with 3 degrees of freedom.

The multiple number of drivers 22a, 22b, 22c and interlocking parts 62a, 62b, 62c may be joined, respectively, to correspond with each other, by way of the multiple number of rods 60a, 60b, 60c, and the driving forces generated (or transferred) according to the manipulation of the drivers 22 may be transferred through the rods 60 to the effector 50, which may then perform various maneuvers required for surgery.

As described above, an instrument according to this embodiment may use a set of rods 60 instead of a separate shaft member, to minimize the diameter of the instrument.

While the multiple number of rods 60a, 60b, 60c are joined with the multiple number of interlocking parts 62a, 62b, 62c to correspond with each other, when one of the rods 60a is operated to move an interlocking part 62a, the other rods 60b, 60c and the interlocking parts 62b, 62c joined to these rods 60b, 60c may support the effector 50 such that the whole of the effector 50 does not move and remains secured to a particular position. Thus, when a rod 60a is operated, only the interlocking part 62a joined to the rod 60a may move.

It is also possible to install a separate securing rod (not shown) in the center or use one or some of the rods 60a, 60b, 60c as a securing rod. Then, the securing rod may support the effector 50 such that the whole of the effector 50 remains secured to a particular position without moving, while the remaining rods may operate to move the respective interlocking parts joined to the rods.

For the purpose of joining the other ends of the rods 60 to the multiple interlocking parts 62 included in the effector 50, a pair of linking devices that mate with each other can be formed on an interlocking part 62 and the other end of a rod 60. Various types of linking device can be applied, examples of which may include forming the interlocking part 62 as an indentation that includes a detent curb and forming the other end of the rod 60 as a hook that is inserted into the indentation and caught on the detent curb; forming the other end of the rod 60 and the interlocking part 62 as a joint, such as a tongue and groove joint, etc.; and attaching a pair of magnets to the other end of the rod 60 and the interlocking part 62.

According to this embodiment, each pair of an interlocking part 62 and a rod 60 may be joined corresponding with each other, and to this end, each pair of linking devices formed on the other end of a rod 60 and an interlocking part 62 can be formed with a different shape and/or structure for each rod 60 (each interlocking part 62). Considering an example where a first rod 60a is to be joined with a first interlocking part 62a and a second rod 60b is to be joined with a second interlocking part 62b, the linking device for the first rod and interlocking part can have a different shape and/or structure from the linking device for the second rod and interlocking part (for example, by forming the first linking device with a square cross section and forming the second linking device with a triangular cross section). Thus, each of the rods 60 and each of the interlocking parts 62 may mate with each other, i.e. a particular rod 60 may be joined with only its counterpart interlocking part 62.

However, it is not imperative that this joining of the interlocking parts 62a, 62b, 62c and rods 60a, 60b, 60c to correspond with each other be implemented by providing different shapes or structures for the linking devices. It is also possible to join the multiple interlocking parts 62a, 62b, 62c and rods 60a, 60b, 60c to each other randomly, and afterwards match the movable parts of the effector 50 with the respective drivers required for manipulation, by identifying which rod 60 is joined to which interlocking part 62.

Various methods can be applied for identifying which rod 60 is joined to which interlocking part 62, where some examples include a method of manually configuring the settings after the effector 50 is joined, and a method of forming electrical contacts on each of the interlocking parts 62 and having the driving part 20 check the ID's of the respective interlocking parts 62 from electrical signals transferred through the rods 60 joined to the interlocking parts 62.

In cases where an instrument according to this embodiment is mounted on a surgical robot for usage, the above matching between the effector 50 and the drivers 22 can be implemented using software within the system for driving the surgical robot. In such cases, the matching settings can be modified according to the requirements of the user, to reconfigure which part of the effector 50 will be operated by which driver 22.

To invade the skin of the patient, a rod 60 according to this embodiment can be formed such that its tip has a needle-like structure. In other words, a needle (not shown) for invasion can be mounted on the other end of the rod 60. If such is the case, a rod 60 according to this embodiment can be structured to have an insulative element coating a conductive element, with the tip of the conductive element electrically connected to a needle, in order that the rod may be used for transferring electrical signals, as described above, or in order that the rod may be used for electrosurgery, as described below.

A surgical instrument according to this embodiment can also be used for electrosurgery. That is, one or more cables (not shown) can be included in addition to the multiple number of rods 60 described above, where the cables may electrically connect the driving part 20 with the effector 50, so that the tip of the effector 50 may be used as an electrosurgical device.

An electrosurgical device can be utilized for stopping blood loss in a vein, cutting tissue, removing small polyps, etc., using a probe equipped with a metal cap or a metal wire that is heated to high temperatures by electricity, as well as for cutting or coagulating tissue using various types of RF waves.

For example, by adding a cable that includes a conductive element coated with an insulative element and electrically connecting the conductive element of the cable to the tip portion of the effector 50, the tip portion of the effector 50 can be supplied with electrical power from the cable to function as an electrosurgical device.

FIG. 32 is a flowchart illustrating a method of setting a surgical instrument according to an embodiment of the present invention. This embodiment relates to a method of setting the instrument described above for use on the surgical site.

First, a separate detached effector module 50 may be provided (P10). An effector 50 according to this embodiment can include a multiple number of interlocking parts 62, as described above, where each part of the effector 50 may be operated according to the manipulation on the interlocking parts 62.

Next, the body of the instrument may be provided, which includes a multiple number of rods 60 joined to the driving part 20 (P20). As already described above, the multiple number of rods 60 may be joined in one end to a multiple number of drivers 22, and each of the rods 60 may be operated in accordance with a manipulation on the respective driver 22.

Next, the other ends of the multiple rods 60 may be joined respectively to the multiple interlocking parts 62 (P30). As described above, the multiple rods 60 and multiple interlocking parts 62 may be joined respectively in correspondence with each other, and for this purpose, each pair of linking devices for a rod 60 and an interlocking part 62 can be formed with a different shape and/or structure for each rod 60, or the rods 60 and the interlocking parts 62 can be joined randomly and matched for correspondence afterwards.

After thus providing the effector module 50 and the instrument body separately and joining them together, the drivers 22 of the driving part 20 may be manipulated to operate the respective parts of the effector 50 (P40). In this way, the setting procedures may be completed for performing a maneuver required for surgery by manipulating the surgical instrument.

When the surgical instrument is set and used according to this embodiment, the separate, detached effector module 50 may be inserted into the surgical site, and the rods 60 may invade the surgical site, after which the effector 50 and the rods 60 may be joined inside the surgical site. Since the surgery can be conducted by inserting the instrument after making an incision in the surgical site of a size that does not require suturing, there may be no scar left in the surgical site, and the laparoscopic surgery may be performed with a greater level of safety.

### INDUSTRIAL APPLICABILITY

While the present invention has been described with reference to particular embodiments, it will be appreciated by those skilled in the art that various changes and modifications can be made without departing from the spirit and scope of the present invention, as defined by the claims appended below.

1. A surgical instrument having an effector joined to one end thereof and a driving part joined to the other end thereof, the effector configured to engage a surgical site, the driving part configured to operate the effector, the surgical instrument comprising:
a first shaft having one end thereof joined with the driving part and extending along a first lengthwise direction;
a second shaft extending along a second lengthwise direction forming a particular angle with the first lengthwise direction, the second shaft having one end thereof joined with the other end of the first shaft such that the second shaft is rotatable about an axis following the second lengthwise direction, the second shaft having the other end thereof joined with the effector; and
a rotatable roller part at a joint part where the first shaft and the second shaft are joined, the roller part supporting a wire connecting the driving wheel and the effector.

2. The surgical instrument according to claim 1, wherein the second shaft is rotatably bearing-joined with the first shaft.

3. The surgical instrument according to claim 1, wherein the length of the first shaft is longer than that of the second shaft.

4. The surgical instrument according to claim 1, wherein the joint part where the first shaft and the second shaft are joined is positioned inside the skin of the surgery patient.

5. The surgical instrument according to claim 1, the surgical instrument mounting on a front end of a surgical robot arm, the surgical robot arm including an actuator, wherein the driving part is a coupler comprising a driving wheel, the driving wheel configured to operate by a driving force transferred from the actuator.

6. The robotic surgical instrument according to claim 5, wherein the second shaft is orthogonal to the first shaft.

7. The robotic surgical instrument according to claim 5, wherein the second shaft is configured to rotate in correspondence with an operation of the driving wheel.

8. The robotic surgical instrument according to claim 5, wherein the second shaft is configured to rotate by a wire joined with the driving wheel and the second shaft.

9. The robotic surgical instrument according to claim 5, wherein the effector is manipulated in correspondence with an operation of the driving wheel.

10. The robotic surgical instrument according to claim 5, wherein the first shaft and the second shaft have different lengths.

11. The robotic surgical instrument according to claim 5, wherein the second shaft is rotatably bearing-joined with the first shaft.

12. (canceled)

13. The robotic surgical instrument according to claim 5, wherein the driving wheel is shaped as a circular disk and is configured to clutch onto the actuator to receive a driving force transferred therefrom.

14. The robotic surgical instrument according to claim 5, further comprising a bending part having a bendable form, the bending part positioned between the second shaft and the effector.

15. The robotic surgical instrument according to claim 14, wherein the driving wheel has a wire joined thereto, the wire configured to apply a tensional force such that the bending part is bent in a particular direction.

16-79. (canceled)
